(19) United States
(12) Patent Application Publication
(10) Pub. No.: US 2009/0197246 A1
(43) Pub. Date: Aug. 6, 2009

(54) Haplotypes and polymorphisms linked to human thiopurine s-methyltransferase deficiencies

(76) Inventor: Udo Stropp, Haan (DE)

Correspondence Address: SIEMENS CORPORATION;INTELLECTUAL PROPERTY DEPARTMENT, 170 WOOD AVENUE SOUTH, ISELIN, NJ, 08830, US

(21) Appl. No.: 10/585,474

(22) PCT Filed: Jan. 7, 2005
(86) PCT No.: PCT/EP05/00064

§ 371 Date: Aug. 15, 2008

(30) Foreign Application Priority Data
Jan. 10, 2004 (EP) 04000398.0

(51) Int. Cl. C12Q 1/68; C07H 21/04

(52) U.S. Cl. 435/6; 536/23.2

(57) ABSTRACT

Haplotypes and polymorphisms of thiopurine S-methyltransferase (TPMT) are described that are linked to TPMT deficiencies which can cause potentially fatal toxicity when patients are treated with thiopurines like mercaptopurine, azathioprine, or thioguanine. The mutant alleles as well as PCR fragments, kits and methods for assaying the TPMT genotype of individual patients are disclosed. Furthermore, algorithms are disclosed that combine the genotypes of a set of single nucleotide polymorphisms to haplotypes that give a distinct information about the TPMT phenotype.

### BACKGROUND OF THE INVENTION

1. Field of the Invention

The present invention is in the field of cancer and immunosuppressive therapeutics, diagnostics, and drug metabolism. In particular, the present invention relates to characterization of the genetic basis for thiopurine methyltransferase deficiency. A number of single nucleotide polymorphisms are, at least in part, responsible for severe hematopoietic toxicity in cancer, Crohn's disease, autoimmune diseases (like rheumatoid arthritis or lupus erythematodes), multiple sclerosis or organ transplant recipient patients who are treated with standard dosages of 6-mercaptopurine, 6-thioguanine or azathioprine (thiopurines in general or other drugs that are substrates of the TPMT enzyme).

2. Related Art

Thiopurine methyltransferase (TPMT, E.C. 2.1.1.67) is a cytoplasmic enzyme that preferentially catalyzes the S-methylation of aromatic and heterocyclic sulfhydryl compounds, including the anticancer agents 6-mercaptopurine (6MP) and 6-thioguanine, and the immunosuppressant azathioprine collectively termed as thiopurines, TPMT activity exhibits genetic polymorphism, with approximately 90% of Caucasians and African-Americans having high TPMT activity, 10% intermediate activity (due to heterozygosity), and 0.3% inheriting TPMT-deficiency as an autosomal recessive trait. (Weinshilbourn, R. M. and Sladek, S. L., Am. J. Hum. Genet. 32:651-662 (1980); McLeod, H. L. et al., Clin. Pharmacol. Ther. 55:15-20 (1994)). TPMT activity can be measured in erythrocytes, as the level of TPMT activity in human liver, kidney, lymphocytes and leukemic lymphoblast correlates with that in erythrocytes (Van Loon, J. A. and Weinshilbourn, R. M., Biochem. Genet. 20:637-658 (1982); Szumlanski, C. L., et al., Pharmacogenetics 2:148-159 (1992); McLeod, H. L. et al., Blood 85:1897-1902 (1995)).

Mercaptopurine, thioguanine, and azathioprine are prodrugs with no intrinsic activity, requiring intracellular conversion to thioguanine nucleotides (TGN), with subsequent incorporation into DNA, as one mechanism of their antiproliferative effect (Lennard, L., Eur. J. Clin. Pharmacol 43:329-339 (1992)). Alternatively, these drugs are metabolized to 6-methyl-mercaptopurine (MeMP) or 6-methyl-thioguanine (MeTG) by TPMT or to 6-thiouric acid (6TU) by xanthine oxidase; MeMP, MeTG, and 6TU are inactive metabolites. Thus, metabolism of 6 MP, azathioprine, or thiaguanine by TPMT shunts drug away from the TGN activation pathway. Clinical studies with 6MP and azathioprine have established an inverse correlation between erythrocyte TPMT activity and erythrocyte TGN accumulation, indicating that patients who less efficiently methylate these thiopurines have more extensive conversion to thioguanine nucleotides (Lennard, L., et al., Lancet 336:225-229 (1990); Lennard, L. et al., Clin. Pharmacol. Ther. 46:149-154 (1989)). Moreover, patients with TPMT deficiency accumulate significantly higher erythrocyte TGN if treated with standard dosages of 6 MP or azathioprine, leading to severe hematopoietic toxicity, unless the thiopurine dosage is lowered substantially (e.g. 8-15 fold reduction) (Evans, W. E., et al., J. Pediatr. 19:985-989 (1991); McLeod, H. L., et al., Lancet 341:1151 (1993); Lennard, L., et al., Arch. Dis. Child. 69:577-579 (1993)) or the converting enzyme hypoxanthine-guanine phosphoribosyltransferase (HGPRT) has a functional defect due to SNPs in promotor, splice or coding regions that reduce activity of HGPRT and by this reduces amounts of thiopurines in the body. The majority of such patients are identified only after experiencing severe toxicity, even though prospective measurement of erythrocyte TPMT activity has been advocated by some (Lennard, L. et al., Clin. Pharmacol. Ther. 41:18-25 (1987)).

Unfortunately, TPMT assays are not widely available and newly diagnosed patients with leukemia or organ transplant recipients are frequently given erythrocyte transfusions, precluding measurement of their constitutive TPMT activity before thiopurine therapy is initiated. Alternatively, several mutant alleles responsible for TPMT deficiency have been described and the relationship between TPMT geno- and phenotype has been most clearly defined for the clinically relevant TPMT alleles *2, *3A and *3C (represented in this file by reference SNPs 44, 47, and 50 respectively) in patients and healthy subjects (Evans et al. J. Clin. Oncol. 19 (2001), 2293-2301, Evans et al. U.S. Pat. No. 5,856,095). Whereupon the heterocygote form of the SNPs correlate to a deficient TPMT activity (reduced activity) and the mutant form of the three SNPs correlate to a more deficient TPMT activity (very reduced or very low activity, sometimes absent activity). Although the several mutant alleles are known to be associated with intermediate or low activity, molecular diagnosis by genotyping can predict the TPMT phenotype only to 85-95% (McLeod, Leukemia 14 (2000), 567-572; Yates, Ann. Intern. Med. 126 (1997), 608-614).

A further relationship between genotype and phenotype was found in differences in the variable number of tandem repeats (VNTR) within the 5′ untranslated region of the TPMT gene (Alves, S. et al., Clinical Pharmacology and Therapeutics 70 (2001), 165-174. The VNTR is composed of 3 repeat elements A, B, and C, differing in length of the unit core (17 or 18 bp) and in nucleotide sequence. Repeats A and B usually can be repeated in the VNTR 1-6 times, repeat C usually is present only ones in the VNTR. Depending on the number of repeats the expression rate of the TPMT protein differs. There seems to be an inverse correlation between the sum of the number of repeats and the VNTR and the level of TPMT activity but this correlation is not very strong and not well studied.

Thus, means and methods for diagnosing and treating diseases, drug responses and disorders based on dysfunction or dysregulations of TPMT are not reliably available yet and lack the needed sensitivity and specificity of a diagnostic test. Thus, the technical problem underlying the present invention is to comply with the above-specified needs.

Identification of the here described single nucleotide polymorphisms at the TPMT locus together with the here disclosed algorithm for combining the respective genotypes of several single nucleotide polymorphism in a patient to one distinct information about the TPMT phenotype would enable a treating physician to prospectively identify TPMT-deficient patients based on their genotype, prior to treatment with potentially toxic dosages of thiopurines like mercaptopurine, azathioprine or thioguanine.

### SUMMARY OF THE INVENTION

The invention relates to the discovery of single nucleotide polymorphisms in the TPMT gene together with an algorithm that can predict TPMT enzyme deficiencies. The presence of these mutant alleles is directly correlated with potentially fatal hematopoietic toxicity when patients are treated with standard dosages of mercaptopurine, azathioprine, or thioguanine.

Based on the discovery of these single nucleotide polymorphisms together with an algorithm, methods have been developed for detecting these inactivating mutations in genomic DNA isolated from individual patients (subjects), to make a diagnosis of TPMT-deficiency, or to identify heterozygous individuals (i.e., people with one mutant gene and one normal gene), having reduced or total deficient TPMT activity. The present invention, therefore, provides a diagnostic test to identify patients with reduced TPMT activity based on their genotype. Such diagnostic test to determine TPMT genotype of patients is quite advantageous because measuring a patient's TPMT enzyme activity has many limitations. Based on this information, we identified here a set of single nucleotide polymorphisms that are new in this combination. Together with a newly developed algorithm these SNPs are able to predict TPMT activity. These tests involve PCR-based amplification of a region of the TPMT gene where the single nucleotide polymorphisms of interests are found. Following amplification, the amplified fragment is assayed for the presence or absence of the specific single nucleotide polymorphisms of interest. Although much of these assays can be done “by hand”, e.g. sequencing oligonucleotide PCR primers, using a thermocycler and protocol to assay for the presence or absence of a single nucleotide polymorphism, automated procedures and kits are designed that contain all the reagents, primers, solutions, et cetera for the genotyping test to facilitate the procedure for use in general clinical laboratories such as those found in a typical hospital, clinic or commercial reference labs.

A preferred embodiment of the present invention relates to the presence of a highly homologues pseudogene in the human genome. Whenever primers were designed to be allele-specific for the TPMT gene we compared both sequences (TPMT gene and pseudogene with bioinformatics programs like MegAlign™ (from DNA Star or other programs) to identify sequences that are unique to the TPMT gene. These are for example the introns of the TPMT gene where allele specific primers for the TPMT gene can be located. For the few differences between the exons of the TPMT gene and the pseudogene primers are located in such a way that the 3′ part of the primer ends exactly on the TPMT gene where there is a difference between the two genes.

In particular, the invention relates to isolated polynucleotide molecules comprising one or more mutant alleles of thiopurine S-methyltransferase (TPMT) or a fragment thereof, which is at least ten consecutive bases long and contains one or more single nucleotide polymorphisms. The single nucleotide polymorphisms are summarized in Table 1.

An aspect of the invention relates to polynucleotide molecules complementary to any one of the polynucleotide molecules described above.

A different aspect of the invention relates to a diagnostic assay for determining thiopurine S-methyl-transferase (TPMT) genotype of a person which comprises isolating nucleic acid from said person, amplifying for a thiopurine S-methyltransferase (TPMT) PCR fragment from said nucleic acid, which includes at least one preferably two or three and in an other aspect more than three of SNPs 1-41 of Table 1, thereby obtaining an amplified fragment. The size of the amplified fragment needs only be large enough so that it is detectable and useful for the genotyping methods described in this file. A preferred range of the amplified fragment size is from 14 nucleotides to several hundreds, more preferably from 75 to 400, and most preferably from 80 to 260.

A further aspect of the invention relates to an isolated polynucleotide molecule having one, two or more SNPs on one or more fragments. Moreover, the invention relates to an isolated polynucleotide molecule complementary to the polynucleotide molecules having a sequence of SNPs 1-41 of Table 1.

An other preferred aspect of the invention relates to genotyping of the amplified fragments with methods described in this file but are not limited to these examples.

An other preferred aspect of the invention is to sequence the VNTR region to identify the number of A, B, and C repeats that correlate to TPMT activity.

Yet another aspect of the invention combines information about the TPMT genotype and the HGPRT genotypes. As inactivating SNPs of the HGPRT gene will produce less, no or deficient HGPRT enzyme, there will be less toxic intermediates produced when a patient is under thiopurine therapy and the treating physician could adjust the dosage of thiopurines in a therapy scheme more precisely.

### DETAILED DESCRIPTION OF THE PREFERRED EMBODIMENTS

The autosomal recessive trait of thiopurine S-methyltransferase (TPMT) deficiency is associated with potentially fatal hematopoietic toxicity when patients are treated with standard dosages of mercaptopurine, azathioprine or thioguanine (thiopurines in general or other drugs that are substrates of the TPMT enzyme). A number of different single nucleotide polymorphisms in the TPMT gene are described herein that we found to be associated to TPMT deficiencies either alone or preferably in combinations (SNPs 1-41 of Table 1).

Based on the sequence of the mutant alleles provided herein, PCR primers are constructed that are complementary to the region of the mutant allele encompassing the single nucleotide polymorphism. A primer consists of a consecutive sequence of polynucleotides complementary to a region in the allele encompassing the position which is mutated in the mutant allele but that does not amplify the pseudogene. PCR primers complementary to a region in the wild-type allele corresponding to the mutant PCR primers are also made to serve as controls in the diagnostic methods of the present invention. The size of these PCR primers ranges anywhere from five bases to hundreds of bases. However, the preferred size of a primer is in the range from 10 to 40 bases, most preferably from 14 to 32 bases.

To amplify the region of the genomic DNA of the individual patient who may be a carrier for the mutant allele, primers to one or both sides of the targeted position, i.e. the SNPs of Table 1, are made and used in a PCR amplification reaction, using known methods in the art (e.g. Massachusetts General Hospital & Harvard Medical School, Current Protocols In Molecular Biology, Chapter 15 (Green Publishing Associates and Wiley-Interscience 1991) and the primers and probes of Table 2. For example for SNP1 the primers SP900295F and SP900295R are used. For the preferred protocols and methods see the Materials and Methods section and Examples.

According to the method of the present invention, once an amplified specific TPMT fragment is obtained (without amplifying the pseudogene), it can be analyzed in several ways to determine whether the patient has one or more of the here described mutant alleles of the TPMT gene. For example, the amplified fragment can be simply sequenced and its sequence compared with the wild-type cDNA sequence of TPMT. If the amplified fragment contains one or more of the single nucleotide polymorphisms described in the present invention and/or the VNTR contains a higher number of repeats A and/or B (for example 3 or more B repeats), the patient is likely to have TPMT-deficiency or be a heterozygote (i.e., reduced activity) and therefore, develop hematopoietic toxicity when treated with standard amounts of mercaptopurine, azathioprine, or thioguanine. Alternatively, a combination of PCR fragment amplification and TaqMan or other genotyping analysis is used to determine TPMT genotype of the individual.

In a preferred embodiment of the invention, a fragment of the genomic DNA of the patient is amplified by TaqMan (Lee et al., Nucleic Acids Research 1993, 21: 3761-3766) analysis using the primers and probes of Table 2 of a respective SNP.

To determine whether the individual is homozygous or heterozygous for TPMT, the mutation sites on the genomic DNA are amplified separately by using wild-type and mutant primers. If only a wild-type or a mutant-type fragment is amplified, the individual is homozygous for the wild-type or the particular mutant-type TPMT. However, presence of more than one type of fragment indicates that the individual is heterozygous for TPMT allele.

An example of a diagnostic assay that is carried out according to the present invention to determine the TPMT genotype of a person is as follows. This example is provided for illustrative purposes and is not meant to be limiting.

Tissue containing DNA (e.g., not red blood cells) from the subject is obtained. Examples of such tissue include white blood cells, mucosal scrapings of the lining of the mouth, epithelial cells, et cetera. Genomic DNA of the individual subject is isolated from this tissue by the known methods in the art, such as phenol/chloroform extraction or commercially available kits like QiaAmp™ DNA kits from Qiagen, Hilden, Germany. An aliquot of the genomic DNA of the subject can be used for PCR amplification of the TPMT gene. PCR primers encompassing the SNPs 1-50 are listed in Table 2 and are marked with an F or R in the ID name (forward and reverse primer) For each specific SNP one primer pair is chosen for example SNP1 can be amplified with SP900295 F and SP900295R. The listed primers are examples for amplification, other primers can be designed by those skilled in the art. Next, the amplicons are analyzed by the various methods described above, which include Taqman analysis, sequencing, mutation-specific amplification, Pyrosequencing™, or other methods that are known to those in the art to measure genotypes.

Hence, an efficient and simple method of obtaining information regarding the TPMT genotype in the patient is now made available which aids the physician in choosing the therapeutic modality for the patient.

### DEFINITIONS

For convenience, the meaning of certain terms and phrases employed in the specification, examples, and appended claims are provided below. Moreover, the definitions by itself are intended to explain a further background of the invention.

The term “algorithm” in this file refers to a sequential analyzing of a number of SNPs in their respective genotypes and defines which genotype of each SNP will have a predictive meaning for TPMT deficiency. For clarification an example is given for 4 SNPs:

| | SNP- | | | |
|---|---|---|---|---|
| | A | SNP-B | SNP-C | SNP-D |
| Polymorphism | G/A | G/A | C/T | A/C |
| Algotrihm1 | GG | +G/A or AA | +CC | +CC |
| Algotrihm2 | GG | — | +CC | +AC |

### Results:

Algorithm1 (combination of 4 SNPs) predicts i.e. reduced enzymatic activity when SNP-A is GG and SNP-B is G/A or AA and SNP-C is CC and SNP-D is CC.
Algorithm2 (combination of 3 SNPs) predicts i.e. total deficient activity when SNP-A is GG and SNP-C is CC and SNP-D is AC.
Identified algorithms are called in this file haplotypes (haplotype 1, 2 etc.)

The term “allele”, which is used interchangeably herein with “allelic variant” refers to alternative forms of a gene or portions thereof. Alleles occupy the same locus or position on homologous chromosomes. When a subject has two identical alleles of a gene, the subject is said to be homozygous for the gene or allele. When a subject has two different alleles of a gene, the subject is said to be heterozygous for the gene. Alleles of a specific gene can differ from each other in a single nucleotide, or several nucleotides, and can include substitutions, deletions, and insertions of nucleotides. An allele of a gene can also be a form of a gene containing a mutation.

The term “allelic variant of a polymorphic region of a gene” refers to a region of a gene having one of several nucleotide sequences found in that region of the gene in other individuals.

“Homology” or “identity” or “similarity” refers to sequence similarity between two peptides or between two nucleic acid molecules. Homology can be determined by comparing a position in each sequence, which may be aligned for purposes of comparison. When a position in the compared sequence is occupied by the same base or amino acid, then the molecules are homologous at that position. A degree of homology between sequences is a function of the number of matching or homologous positions shared by the sequences.

The term “pseudogene” refers to sequences that have a high homology to identified genes and are generally untranscribed and untranslated due to non-functional promoters, missing start codons or other defects. Most Pseudogenes are intronless and represent mainly the coding sequence of the parent gene. For some cases it has been shown that in different organisms or tissues functional activation may occur.

The term “intronic sequence” or “intronic nucleotide sequence” refers to the nucleotide sequence of an intron or portion thereof.

The term “locus” refers to a specific position in a chromosome. For example, a locus of a gene refers to the chromosomal position of the gene.

The term “molecular structure” of a gene or a portion thereof refers to the structure as defined by the nucleotide content (including deletions, substitutions, additions of one or more nucleotides), the nucleotide sequence, the state of methylation, and/or any other modification of the gene or portion thereof.

The term “mutated gene” refers to an allelic form of a gene, which is capable of altering the phenotype of a subject having the mutated gene relative to a subject, which does not have the mutated gene. If a subject must be homozygous for this mutation to have an altered phenotype, the mutation is said to be recessive. If one copy of the mutated gene is sufficient to alter the genotype of the subject, the mutation is said to be dominant. If a subject has one copy of the mutated gene and has a phenotype that is intermediate between that of a homozygous and that of a heterozygous (for that gene) subject, the mutation is said to be co-dominant.

As used herein, the term “nucleic acid” refers to polynucleotides such as deoxynbonucleic acid (DNA), and, where appropriate, ribonucleic acid (RNA). The term should also be understood to include, as equivalents, derivatives, variants and analogs of either RNA or DNA made from nucleotide analogs, including peptide nucleic acids (PNA), morpholino oligonucleotides (J. Summerton and D. Weller, Antisense and Nucleic Acid Drug Development 7:187 (1997)) and, as applicable to the embodiment being described, single (sense or antisense) and double-stranded polynucleotides. Deoxyribonucleotides include deoxyadenosine, deoxycytidine, deoxyguanosine, and deoxythymidine. For purposes of clarity, when referring herein to a nucleotide of a nucleic acid, which can be DNA or an RNA, the term “adenosine”, “cytidine”, “guanosine”, and “thymidine” are used. It is understood that if the nucleic acid is RNA, a nucleotide having a uracil base is uridine.

The term “polymorphism” refers to the coexistence of more than one form of a gene or portion thereof. A portion of a gene of which there are at least two different forms, i.e., two different nucleotide sequences, is referred to as a “polymorphic region of a gene”. A polymorphic region can be a single nucleotide, the identity of which differs in different alleles. A polymorphic region can also be several nucleotides long.

A “polymorphic gene” refers to a gene having at least one polymorphic region.

To describe a “polymorphic site” in a nucleotide sequence often there is used an “ambiguity code” that stands for the possible variations of nucleotides in one site. The list of ambiguity codes is summarized in the following table:

Ambiguity Codes
(IUPAC Nomenclature)

| Code | Nucleotides |
|---|---|
| B | c/g/t |
| D | a/g/t |
| H | a/c/t |
| K | g/t |
| M | a/c |
| N | a/c/g/t |
| R | a/g |
| S | c/g |
| V | a/c/g |
| W | a/t |
| Y | c/t |

For example, a “R” in a nucleotide sequence means that either an “a” or a “g” nucleotide could be at that position.

The terms “protein”, “polypeptide” and “peptide” are used interchangeably herein when referring to a gene product.

A “regulatory element”, also termed herein “regulatory sequence is intended to include elements which are capable of modulating transcription from a basic promoter and include elements such as enhancers and silencers. The term “enhancer”, also referred to herein as “enhancer element”, is intended to include regulatory elements capable of increasing, stimulating, or enhancing transcription from a basic promoter. The term “silencer”, also referred to herein as “silencer element” is intended to include regulatory elements capable of decreasing, inhibiting, or repressing transcription from a basic promoter. Regulatory elements are typically present in 5′ flanking regions of genes. However, regulatory elements have also been shown to be present in other regions of a gene, in particular in introns. Thus, it is possible that genes have regulatory elements located in introns, exons, coding regions, and 3′ flanking sequences. Such regulatory elements are also intended to be encompassed by the present invention and can be identified by any of the assays that can be used to identify regulatory elements in 5′ flanking regions of genes.

As used herein, the term “specifically hybridizes” or “specifically detects” refers to the ability of a nucleic acid molecule of the invention to hybridize to at least approximately 6, 12, 20, 30, 40, 50, 60, 70, 80, 90, 100, 110, 120, 130 or 140 consecutive nucleotides of either strand of a gene.

The term “wild-type allele” refers to an allele of a gene which, when present in two copies in a subject results in a wild-type phenotype. There can be several different wild-type alleles of a specific gene, since certain nucleotide changes in a gene may not affect the phenotype of a subject having two copies of the gene with the nucleotide changes.

“Adverse drug reaction” (ADR) as used herein refers to an appreciably harmful or unpleasant reaction, resulting from an intervention related to the use of a medicinal product, which

predicts hazard from future administration and warrants prevention or specific treatment, or alteration of the dosage regimen, or withdrawal of the product. In it's most severe form an ADR might lead to the death of an individual.

The term “drug response” is intended to mean any response that a patient exhibits upon drug administration. Specifically drug response includes beneficial, i.e. desired drug effects, ADR or no detectable reaction at all. More specifically the term drug response could also have a qualitative meaning, i.e. it embraces low or high beneficial effects, respectively and mild or severe ADR, respectively. An individual drug response includes also a good or bad metabolizing of the drug, meaning that “bad metabolizers” accumulate the drug in the body and by this could show side effects of the drug due to accumulative overdoses.

The term “haplotype” as used herein refers to a group of two or more SNPs that are functionally and/or spatially linked. Haplotypes of this file are described by an algorithm. Haplotypes are expected to give better predictive/diagnostic information than a single SNP.

The term “haplotype block” as used herein refers to the observable linkage of SNPs between recombination hot spots the locations where homologous recombination between maternal and paternal chromosomes takes place during meiosis. Hot spots on chromosomes have distances between roughly 5000 to 100,000 base pairs. SNPs between hot spots are in higher linkage than SNPs outside the blocks. Haplotypes blocks can experimentally be identified through genotyping a number of neighboring SNPs on a chromosome and analyzing which SNPs are linked (have a comparable genotype pattern).

The term “deficient TPMT activity” in a person can mean absent or very low TPMT activity or it can mean intermediate activity, which is between very low, and the low-end of normal TPMT activity.

### Diagnostic and Prognostic Assays

The present invention provides methods for determining the molecular structure of at least one polymorphic region of a gene, specific allelic variants and haplotypes of said polymorphic region being associated with TPMT deficiencies. In one embodiment, determining the molecular structure of a polymorphic region of a gene comprises determining the identity of the allelic variant. A polymorphic region of a gene, of which specific alleles are associated with TPMT deficiencies can be located in an exon, an intron, at an intron/exon border, or in the promoter or other 5′ or 3′ flanking regions of the coding sequence of the gene.

In case of analyzing TPMT gene polymorphisms a TPMT gene-specific amplification is recommended to omit interference of sequences from the TPMT pseudogene as discussed above.

The invention provides methods for determining whether a subject has a functional defect in metabolizing thiopurines or structural analogues that are metabolized by TPMT.

In preferred embodiments, the methods of the invention can be characterized as comprising detecting, in a sample of cells from the subject, the presence or absence of specific allelic variants of one or more polymorphic regions of a gene. The allelic differences can be: (i) a difference in the identity of at least one nucleotide or (ii) a difference in the number of nucleotides, which difference can be a single nucleotide or several nucleotides.

Due to the presence of a TPMT pseudogene in the human genome, which is highly homologues to the exons of the TPMT gene most detection methods, need first to amplify at least a portion of a gene prior to identifying the allelic variant. An example is given in the following: Primers for gene-specific amplification have to be located in sequences on the gene of interest that show no homology to the pseudogene, for example the intron sequences of the gene of interest or other sequences that are unique to the gene of interest. Those skilled in the art find those unique sequences through pairwise alignment of homologous sequences of the gene of interest with the help of bioinformatics tools like MegAlign™ (DNA Star) or ClustalW™ from the Wisconsin Genetics Computer Group or other programs. Amplification of the gene fragments can be performed, e.g., by PCR and/or by ligase chain reaction (LCR), according to methods known in the art. In one embodiment, genomic DNA of a cell is exposed to two PCR primers and amplification for a number of cycles sufficient to produce the required amount of amplified DNA. In preferred embodiments, the primers are located between 40 and 350 base pairs apart. Preferred primers for amplifying gene fragments of genes of this file are listed in Table 2 in the Examples.

A preferred detection method is allele specific hybridization using probes overlapping the polymorphic site and having about 5, 10, 20, 25, or 30 nucleotides around the polymorphic region. Examples of probes for detecting specific allelic variants of the polymorphic region are probes comprising a nucleotide sequence set forth in any of SNPs 1-41 in Table 1. In a preferred embodiment of the invention, several probes capable of hybridizing specifically to allelic variants are attached to a solid phase support, e.g., a “chip”. Oligonucleotides can be bound to a solid support by a variety of processes, including lithography. For example a chip can hold up to 250,000 oligonucleotides (GeneChip, Affymetrix). Mutation detection analysis using these chips comprising oligonucleotides, also termed “DNA probe arrays” is described e.g., in Cronin et al. (1996) Human Mutation 7:244 and in Kozal et al. (1996) Nature Medicine 2:753. In one embodiment, a chip comprises all the allelic variants of at least one polymorphic region of a gene. The solid phase support is then contacted with a test nucleic acid and hybridization to the specific probes is detected. Accordingly, the identity of numerous allelic variants of one or more genes can be identified in a simple hybridization experiment. For example, the identity of the allelic variant of the nucleotide polymorphism of nucleotide G or A at position 16 of SNP1 in Table 1 and that of other possible polymorphic regions can be determined in a single hybridization experiment. In case of TPMT gene analysis prior to hybridization experiments a gene-specific amplification is needed to get rid of the pseudogene sequences which would interfere in hybridization experiments.

Alternative amplification methods include: self sustained sequence replication (Guatelli, J. C. et al., 1990, Proc. Natl. Acad. Sci. U.S.A. 87:1874-1878), transcriptional amplification system (Kwoh, D. Y. et al., 1989, Proc. Natl. Acad. Sci. U.S.A. 86:1173-1177), Q-Beta Replicase (Lizardi, P. M. et al., 1988, Bio/Technology 6:1197), or any other nucleic acid amplification method, followed by the detection of the amplified molecules using techniques well known to those of skill in the art. These detection schemes are especially useful for the detection of nucleic acid molecules if such molecules are present in very low numbers.

In one embodiment, any of a variety of sequencing reactions known in the art can be used to directly sequence at least a portion of a gene and detect allelic variants, e.g., mutations, by comparing the sequence of the sample sequence with the corresponding wild-type (control) sequence. Exemplary sequencing reactions include those based on techniques developed by Maxam and Gilbert (Proc. Natl Acad Sci USA (1977) 74:560) or Sanger (Sanger et al (1977) Proc. Nat. Acad. Sci 74:5463). It is also contemplated that any of a variety of automated sequencing procedures may be utilized when performing the subject assays (Biotechniques (1995) 19:448), including sequencing by mass spectrometry (see, for example, U.S. Pat. No. 5,547,835 and international patent application Publication Number WO 94/16101, entitled DNA Sequencing by Mass Spectrometry by H. Koster, U.S. Pat. No. 5,547,835 and international patent application Publication Number WO 94/21822 entitled “DNA Sequencing by Mass Spectrometry Via Exonuclease Degradation” by H. Koster), and U.S. Pat. No. 5,605,798 and International Patent Application No. PCT/US96/03651 entitled DNA Diagnostics Based on Mass Spectrometry by H. Koster; Cohen et al. (1996) Adv Chromatogr 36:127-162; and Griffin et al. (1993) Appl Biochem Biotechnol 38:147-159). It will be evident to one skilled in the art that, for certain embodiments, the occurrence of only one, two or three of the nucleic acid bases need be determined in the sequencing reaction. For instance, A-track or the like, e.g., where only one nucleotide is detected, can be carried out.

Yet other sequencing methods are disclosed, e.g., in U.S. Pat. No. 5,580,732 entitled “Method of DNA sequencing employing a mixed DNA-polymer chain probe” and U.S. Pat. No. 5,571,676 entitled “Method for mismatch-directed in vitro DNA sequencing”.

In some cases, the presence of a specific allele of a gene in DNA from a subject can be shown by restriction enzyme analysis. For example, a specific nucleotide polymorphism can result in a nucleotide sequence comprising a restriction site which is absent from the nucleotide sequence of another allelic variant.

In other embodiments, alterations in electrophoretic mobility are used to identify the type of gene allelic variant. For example, single strand conformation polymorphism (SSCP) may be used to detect differences in electrophoretic mobility between mutant and wild type nucleic acids (Orita et al. (1989) Proc Natl. Acad. Sci USA 86:2766, see also Cotton (1993) Mutat Res 285:125-144; and Hayashi (1992) Genet Anal Tech Appl 9:73-79). Single-stranded DNA fragments of sample and control nucleic acids are denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In another preferred embodiment, the subject method utilizes heteroduplex analysis to separate double stranded heteroduplex molecules on the basis of changes in electrophoretic mobility (Keen et al. (1991) Trends Genet 7:5).

In yet another embodiment, the identity of an allelic variant of a polymorphic region is obtained by analyzing the movement of a nucleic acid comprising the polymorphic region in polyacrylamide gels containing a gradient of denaturant is assayed using denaturing gradient gel electrophoresis (DGGE) (Myers et al (1985) Nature 313:495). When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing agent gradient to identify differences in the mobility of control and sample DNA (Rosenbaum and Reissner (1987) Biophys Chem 265:1275).

Examples of techniques for detecting differences of at least one nucleotide between 2 nucleic acids include, but are not limited to, selective oligonucleotide hybridization, selective amplification, or selective primer extension. For example, oligonucleotide probes may be prepared in which the known polymorphic nucleotide is placed centrally (allele-specific probes) and then hybridized to target DNA under conditions which permit hybridization only if a perfect match is found (Saiki et al. (1986) Nature 324:163); Saiki et al (1989) Proc. Natl Acad. Sci USA 86:6230; and Wallace et al. (1979) Nucl. Acids Res. 6:3543). Such allele specific oligonucleotide hybridization techniques may be used for the simultaneous detection of several nucleotide changes in different polymorphic regions of gene. For example, oligonucleotides having nucleotide sequences of specific allelic variants are attached to a hybridizing membrane and this membrane is then hybridized with labeled sample nucleic acid. Analysis of the hybridization signal will then reveal the identity of the nucleotides of the sample nucleic acid.

Alternatively, allele specific amplification technology which depends on selective PCR amplification may be used. Oligonucleotides used as primers for specific amplification may carry the allelic variant of interest in the center of the molecule (so that amplification depends on differential hybridization) (Gibbs et al (1989) Nucleic Acids Res. 17:2437-2448) or at the extreme 3′ end of one primer where, under appropriate conditions, mismatch can prevent, or reduce polymerase extension (Prossner (1993) Tibtech 11:238; Newton et al. (1989) Nucl. Acids Res. 17:2503). This technique is also termed “PROBE” for Probe Oligo Base Extension. In addition it may be desirable to introduce a novel restriction site in the region of the mutation to create cleavage-based detection (Gasparini et al (1992) Mol. Cell Probes 6:1).

In another embodiment, identification of the allelic variant is carried out using an oligonucleotide ligation assay (OLA), as described, e.g., in U.S. Pat. No. 4,998,617 and in Landegren, U. et al., Science 241:1077-1080 (1988). The OLA protocol uses two oligonucleotides which are designed to be capable of hybridizing to abutting sequences of a single strand of a target. One of the oligonucleotides is linked to a separation marker, e.g., biotinylated, and the other is detectably labeled. If the precise complementary sequence is found in a target molecule, the oligonucleotides will hybridize such that their termini abut, and create a ligation substrate. Ligation then permits the labeled oligonucleotide to be recovered using avidin, or another biotin ligand. Nickerson, D. A. et al. have described a nucleic acid detection assay that combines attributes of PCR and OLA (Nickerson, D. A. et al., Proc. Natl. Acad. Sci. (U.S.A.) 87:8923-8927 (1990). In this method, PCR is used to achieve the exponential amplification of target DNA, which is then detected using OLA.

Several techniques based on this OLA method have been developed and can be used to detect specific allelic variants of a polymorphic region of a gene. For example, U.S. Pat. No. 5,593,826 discloses an OLA using an oligonucleotide having 3′-amino group and a 5′-phosphorylated oligonucleotide to form a conjugate having a phosphoramidate linkage. In another variation of OLA described in Tobe et al. ((1996) Nucleic Acids Res 24: 3728), OLA combined with PCR permits typing of two alleles in a single microtiter well. By marking each of the allele-specific primers with a unique hapten, i.e. digoxigenin and fluorescein, each LA reaction can be detected by using hapten specific antibodies that are labeled with different enzyme reporters, alkaline phosphatase or horseradish peroxidase. This system permits the detection of the two alleles using a high throughput format that leads to the production of two different colors.

The invention further provides methods for detecting single nucleotide polymorphisms in a gene. Because single nucleotide polymorphisms constitute sites of variation flanked by regions of invariant sequence, their analysis requires no more than the determination of the identity of the single nucleotide present at the site of variation and it is unnecessary to determine a complete gene sequence for each patient. Several methods have been developed to facilitate the analysis of such single nucleotide polymorphisms.

In one embodiment, the single base polymorphism can be detected by using a specialized exonuclease-resistant nucleotide, as disclosed, e.g., in Mundy, C. R. (U.S. Pat. No. 4,656,127). According to the method, a primer complementary to the allelic sequence immediately 3′ to the polymorphic site is permitted to hybridize to a target molecule obtained from a particular animal or human. If the polymorphic site on the target molecule contains a nucleotide that is complementary to the particular exonuclease-resistant nucleotide derivative present, then that derivative will be incorporated onto the end of the hybridized primer. Such incorporation renders the primer resistant to exonuclease, and thereby permits its detection. Since the identity of the exonuclease-resistant derivative of the sample is known, a finding that the primer has become resistant to exonucleases reveals that the nucleotide present in the polymorphic site of the target molecule was complementary to that of the nucleotide derivative used in the reaction. This method has the advantage that it does not require the determination of large amounts of extraneous sequence data.

In another embodiment of the invention, a solution-based method is used for determining the identity of the nucleotide of a polymorphic site. Cohen, D. et al. (French Patent 2,650,840; PCT Appln. No. WO91/02087). As in the Mundy method of U.S. Pat. No. 4,656,127, a primer is employed that is complementary to allelic sequences immediately 3′ to a polymorphic site. The method determines the identity of the nucleotide of that site using labeled dideoxynucleotide derivatives, which, if complementary to the nucleotide of the polymorphic site will become incorporated onto the terminus of the primer.

An alternative method, known as Genetic Bit Analysis or GBA™ is described by Goelet, P. et al. (PCT Appln. No. 92/15712). The method of Goelet, P. et al. uses mixtures of labeled terminators and a primer that is complementary to the sequence 3′ to a polymorphic site. The labeled terminator that is incorporated is thus determined by, and complementary to, the nucleotide present in the polymorphic site of the target molecule being evaluated. In contrast to the method of Cohen et al. (French Patent 2,650,840; PCT Appln. No. WO91/02087) the method of Goelet, P. et al. is preferably a heterogeneous phase assay, in which the primer or the target molecule is immobilized to a solid phase.

Recently, several primer-guided nucleotide incorporation procedures for assaying polymorphic sites in DNA have been described (Komher, J. S. et al., Nucl. Acids. Res. 17:7779-7784 (1989); Sokolov, B. P., Nucl. Acids Res. 18:3671 (1990); Syvanen, A.-C., et al., Genomics 8:684-692 (1990), Kuppuswamy, M. N. et al., Proc. Natl. Acad. Sci. (U.S.A.) 88:1143-1147 (1991); Prezant, T. R. et al., Hum. Mutat. 1:159-164 (1992); Ugozzoli, L. et al., GATA 9:107-112 (1992); Nyren, P. et al., Anal. Biochem 208:171-175 (1993)). These methods differ from GBA™ in that they all rely on the incorporation of labeled deoxynucleotides to discriminate between bases at a polymorphic site. In such a format, since the signal is proportional to the number of deoxynucleotides incorporated, polymorphisms that occur in runs of the same nucleotide can result in signals that are proportional to the length of the run (Syvanen, A.-C., et al., Amer. J. Hum. Genet 52:46-59 (1993)).

For determining the identity of the allelic variant of a polymorphic region located in the coding region of a gene, yet other methods than those described above can be used. For example, using an antibody specifically recognizing the mutant protein in, e.g., immunohistochemistry or immunoprecipitation can perform identification of an allelic variant, which encodes a mutated gene protein. Antibodies to wild-type gene protein are described, e.g., in Acton et al. (1999) Science 271:518 (anti-mouse gene antibody cross-reactive with human gene). Other antibodies to wild-type gene or mutated forms of gene proteins can be prepared according to methods known in the art. Alternatively, one can also measure an activity of a gene protein, such as binding to a lipid or lipoprotein. Binding assays are known in the art and involve, e.g., obtaining cells from a subject, and performing binding experiments with a labeled lipid, to determine whether binding to the mutated form of the receptor differs from binding to the wild-type of the receptor.

If a polymorphic region is located in an exon, either in a coding or non-coding region of the gene, the identity of the allelic variant can be determined by determining the molecular structure of the mRNA, pre-mRNA, or cDNA. The molecular structure can be determined using any of the above described methods for determining the molecular structure of the genomic DNA, e.g., sequencing and SSCP.

The methods described herein may be performed, for example, by utilizing pre-packaged diagnostic kits, such as those described above, comprising at least one probe or primer nucleic acid described herein, which may be conveniently used, e.g., to determine whether a subject is at risk of having TPMT deficiencies which can cause severe side effects when treated with thiopurines or analogues.

Sample nucleic acid for using in the above-described diagnostic and prognostic methods can be obtained from any cell type or tissue of a subject. For example, a subject's bodily fluid (e.g. blood or saliva) can be obtained by known techniques (e.g. venipuncture or swab, respectively) or from human tissues like heart (biopsies, transplanted organs). Alternatively, nucleic acid tests can be performed on dry samples (e.g. hair or skin).

Diagnostic procedures may also be performed in situ directly upon tissue sections (fixed and/or frozen) of patient tissue obtained from biopsies or resections, such that no nucleic acid purification is necessary. Nucleic acid reagents may be used as probes and/or primers for such in situ procedures (see, for example, Nuovo, G. J., 1992, PCR in situ hybridization: protocols and applications, Raven Press, New York).

In addition to methods which focus primarily on the detection of one nucleic acid sequence, profiles may also be assessed in such detection schemes. Fingerprint profiles may be generated, for example, by utilizing a differential display procedure, Northern analysis and/or RT-PCR.

In practicing the present invention, the distribution of polymorphic patterns in a large number of individuals exhibiting particular markers for thiopurine response is determined by any of the methods described above, and compared with the distribution of polymorphic patterns in patients that have been matched for age, ethnic origin, and/or any other statistically or medically relevant parameters, who exhibit quantitatively or qualitatively different status markers. Correlations are achieved using any method known in the art, including nominal logistic regression, chi square tests or standard least squares regression analysis. In this manner, it is possible to establish statistically significant correlations between particular polymorphic patterns and particular thiopurine response statuses (given in p values). It is further possible to establish statistically significant correlations between particular polymorphic patterns and changes in drug response such as, would result, e.g., from particular treatment regimens. In this manner, it is possible to correlate polymorphic patterns with responsivity to particular treatments.

In another embodiment of the present invention two or more polymorphic regions are combined to define so called ‘haplotypes’. Haplotypes are groups of two or more SNPs that are functionally and/or spatially linked. It is possible to combine SNPs that are disclosed in the present invention either with each other or with additional polymorphic regions to form a haplotype. Haplotypes are expected to give better predictive/diagnostic information than a single SNP.

In a preferred embodiment of the present invention a panel of SNPs/haplotypes is defined that predicts drug response. This predictive panel is then used for genotyping of patients on a platform that can genotype multiple SNPs at the same time (Multiplexing). Preferred platforms are e.g. gene chips (Affymetrix) or the Luminex LabMAP™ reader. But also newer developments are under way like planar waveguides or nanoparticles that could be used for multiplex genotyping. Thin film planar waveguides (PWGs) as used by Zeptosens, Witterswil, Switzerland, for example consist of a 150 to 300 nm thin film of a material with high refractive index (e.g. Ta₂O₅ or TiO₂), which is deposited on a transparent support with lower refractive index (e.g. glass or polymer). A parallel laser light beam is coupled into the waveguiding film by a diffractive grating that is etched or embossed into the substrate. The light propagates within this film and creates a strong evanescent field perpendicular to the direction of propagation into the adjacent medium. The field strength decays exponentially with the distance from the waveguide surface, and its penetration depth is limited to about 400 nm. This effect can be utilized to selectively excite only fluorophores located at or near the surface of the waveguide.

For diagnostics applications, specific captures are immobilized on the waveguide surface. The presence of the analyte in a sample applied to a PWG chip is detected using fluorescent reporter molecules attached to the analyte or one of its binding partners in the assay. Upon fluorescence excitation by the evanescent field, excitation and detection of fluorophores is restricted to the sensing surface, whilst signals from unbound molecules in the bulk solution are not detected. Using this technology it is possible to detect polymorphisms in the TPMT gene but one has to be careful in designing the capture probes in respect to the pseudogene (see discussion above on identifying non-homologous sequences between gene and pseudogene.

Alternatively, nanoparticles could be used that emit different fluorescent colors so that a multiplexing can be set-up for several SNP assays in one reaction as discussed for example in (Expert Rev Mol Diagn. 2003; 3(2): 153-61).

The subsequent identification and evaluation of a patient's haplotype can then help to guide specific and individualized therapy.

For example the present invention can identify patients exhibiting genetic polymorphisms or haplotypes which indicate an increased risk for adverse drug reactions. In that case the drug dose should be lowered in a way that the risk for ADR is diminished.

It is self evident that the ability to predict a patient's individual drug response should affect the formulation of a drug, i.e. drug formulations should be tailored in a way that they suit the different patient classes (low/high responder, poor/good metabolizer, and ADR prone patients). Those different drug formulations may encompass different doses of the drug, i.e. the medicinal products contains low or high amounts of the active substance. In another embodiment of the invention the drug formulation may contain additional substances that facilitate the beneficial effects and/or diminish the risk for ADR (Folkers et al. 1991, U.S. Pat. No. 5,316,765).

### Isolated Polymorphic Nucleic Acids, and Probes

The present invention provides isolated nucleic acids comprising the polymorphic positions described herein for human genes. The invention also provides probes, which are useful for detecting these polymorphisms.

In practicing the present invention, many conventional techniques in molecular biology. Such techniques are well known and are explained fully in, for example, Sambrook et al., 2000, Molecular Cloning: A Laboratory Manual, Third Edition, Cold Spring Harbor Laboratory Press, Cold Spring Harbor, N.Y.; DNA Cloning: A Practical Approach, Volumes I and II, 1985 (D. N. Glover ed.); Oligonucleotide Synthesis, 1984, (M. L. Gait ed.); Nucleic Acid Hybridization, 1985, (Hames and Higgins); Ausubel et al., Current Protocols in Molecular Biology, 1997, (John Wiley and Sons); and Methods in Enzymology Vol. 154 and Vol. 155 (Wu and Grossman, and Wu, eds., respectively).

The nucleic acids of the present invention find use as probes for the detection of genetic polymorphisms and as templates for the recombinant production of normal or variant peptides or polypeptides encoded by genes listed in the Examples.

Probes in accordance with the present invention comprise without limitation isolated nucleic acids of about 10-100 bp, preferably 14-75 bp and most preferably 15-25 bp in length, which hybridize at high stringency to one or more of the polymorphic sequences disclosed herein or to a sequence immediately adjacent to a polymorphic position. Furthermore, in some embodiments a full-length gene sequence may be used as a probe. In one series of embodiments, the probes span the polymorphic positions in genes disclosed herein. In another series of embodiments, the probes correspond to sequences immediately adjacent to the polymorphic positions.

### Kits

As set forth herein, the invention provides diagnostic methods, e.g., for determining the identity of the allelic variants of polymorphic regions present in the gene loci of genes disclosed herein, wherein specific allelic variants of the polymorphic region are associated with TPMT deficiencies. In a preferred embodiment, the diagnostic kit can be used to determine whether a subject is at risk suffering severe side effects when treated with thiopurines. This information could then be used, e.g., to optimize treatment of such individuals.

In preferred embodiments, the kit comprises probe and primers, which are capable of hybridizing to a gene and thereby identifying whether the gene contains an allelic variant of a polymorphic region which is associated with TPMT deficiencies. The kit further comprises an algorithm that identifies from a combination of SNPs the grade of TPMT deficiency. The kit preferably further comprises instructions for use in diagnosing a subject having TPMT deficiencies. The probe or primers of the kit can be any of the probes or primers described in this file.

Preferred kits for amplifying a region of a gene comprising a polymorphic region of interest comprise one, two or more primers.

### Material and Methods

Genotyping using the ABI 7700/7900™ instrument (for TaqMan analysis)

Genotyping of patient DNA using the TaqMan (Applied Biosystems/Perkin Elmer) was performed according to the manufacturer's instructions. The TaqMan assay is discussed by Lee et al., Nucleic Acids Research 1993, 21:3761-3766.

### Human Subjects

Whole blood was obtained from a central diagnostic lab as waste material, individual labels on the tubs were removed irreversibly and replaced by a new number. DNA was isolated with commercially available kits (QIAamp DNA Blood Mini Kit) from Qiagen, Hilden, Germany.

### Examples

The following examples are intended to further illustrate certain preferred embodiments of the invention and are not intended to be limiting in nature.

### Genotyping Assays

DNA of ca. 1300 anonymyzed blood samples was genotyped for 50 SNPs that are listed in Table 1. The sequence of each SNP is given in the table with each SNP in the middle of the sequence. The position is also given in numbers where the SNP can be found on the sequence. As a reference the TPMT gene sequence was taken from the NCBI, accession number AL589723, the sequence was reversed and complemented as the coding sequence for the TPMT enzyme is not given in 5′-3′ direction. For all 50 SNPs a TaqMan assay was designed with PCR primers and TaqMan probes for each allele with different dyes as listed in Table 2. The general protocol for using TaqMan is mentioned above. One example protocol is given in Table 3 for describing concentrations of primers and probes, DNA, and other parameters like cycle temperatures and times.

TABLE 1

SNP sequences of SNPs 1-50 including surrounding sequence plus
respective position in Accession number AL589723 (reverse comple-
ment). Nine reference SNPs were included for benchmarking. SNPs 44,
47, and 50 have proven genotype to phenotype correlation (see
above). SNPs 42, 43, 45, 46, 48, and 49 are from a recent patent
application,. WO 03/066892 A1.

| | | | | | SNP Pos. | |
|---|---|---|---|---|---|---|
| SEQ-ID | SNP-ID | Bay-SNP | Position | rs # (NCBI) | in Seq. | Sequence around SNP |
| SEQ-1 | SNP1 | 900295 | 14127 | rs 1011620 | C16T | CTAAGTATTTTTTCTYCTCCT |
| | | | | | | TGCATTACCA |
| SEQ-2 | SNP2 | 900294 | 19328 | rs 942470 | T16C | AAGGCATAGTGTTATYTGAA |
| | | | | | | AGAGAAATTAA |
| SEQ-3 | SNP3 | 900296 | 23375 | rs 1886330 | T16G | ATTTGTTTTCTCGATKTTATT |
| | | | | | | GAACCTTAAC |
| SEQ-4 | SNP4 | 900272 | 23670 | rs 2328212 | C16T | GGTAGATATGGTTGGYTGGA |
| | | | | | | TTTGAGGACAC |
| SEQ-5 | SNP5 | 900297 | 29246 | rs 3806961 | T16C | CAACACCTGCAAGGCYGTGC |
| | | | | | | GGGCTCCTGGC |
| SEQ-6 | SNP6 | 900273 | 29586 | rs 3806962 | C16T | CCTAGCCCGGGAATTYCCCC |
| | | | | | | TTCTTCAGACA |
| SEQ-7 | SNP7 | 900298 | 31089 | rs 2842942 | T16A | TTGTGGGCAGAAATTWTTGT |
| | | | | | | GAAATTTCCCT |
| SEQ-8 | SNP8 | 900338 | 32274 | | A16T | TATACATATTCAGTWAGCTG |
| | | | | | | TAGGATGAC |
| SEQ-9 | SNP9 | 900314 | 33796 | rs 2427790 | A16C | AATAAATAAATAAATMAATC |
| | | | | | | TAGGTTTCCAA |
| SEQ-10 | SNP10 | 900315 | 36499 | rs 3931660 | T16A | TGCACATTTAATTCTWCACA |
| | | | | | | TTTTTGTGTCT |
| SEQ-11 | SNP11 | 900299 | 36905 | rs 2842940 | T16A | TGCTGAGTAAAGTGGWTGTT |
| | | | | | | AGAGACATTCC |
| SEQ-12 | SNP12 | 900300 | 37091 | rs 2518471 | C16T | TCTCAGGTTTACTTCYGAGG |
| | | | | | | CTTGAGTACAC |
| SEQ-13 | SNP13 | 900301 | 37210 | rs 2518472 | A16T | TAATAAAGAATTTTCWAAAC |
| | | | | | | ATCCCCAAGAA |
| SEQ-14 | SNP14 | 900274 | 37420 | rs 3928922 | T16A | AGTGTTCACCTACCAWACAA |
| | | | | | | TTGTCCTAAAA |
| SEQ-15 | SNP15 | 900337 | 37463 | | T16C | TCCTCTTCAGGCTATYAAAG |
| | | | | | | AAGCATTTAG |
| SEQ-16 | SNP16 | 900316 | 37585 | rs 4449636 | C16T | AACAGAATTATCTTGYCTTA |
| | | | | | | ATGATGAATTC |
| SEQ-17 | SNP17 | 900336 | 37646 | | G16T | AAACTCCATTTTCAGKAAAT |
| | | | | | | ACACAGAAAT |
| SEQ-18 | SNP18 | 900317 | 37824 | rs 3898137 | C16T | TTCCCTTTTACATTTYCTGGA |
| | | | | | | TCCTTGTATG |
| SEQ-19 | SNP19 | 900318 | 38079 | rs 7454407 | G16A | GTAATTCTCTACAAARAGAA |
| | | | | | | TTCACTTTAAC |
| SEQ-20 | SNP20 | 900275 | 40232 | rs 2518462 | T16A | ATTTTAGGAAGGCACWTGTT |
| | | | | | | ACATTATAGCA |
| SEQ-21 | SNP21 | 900335 | 41703 | | G16T | GAACTTGGGATACAAKAATT |
| | | | | | | TTTTACAGAG |
| SEQ-22 | SNP22 | 900334 | 41750 | | C16T | AGAAGAACCAATCACYGAA |
| | | | | | | ATTCCTGGAAC |
| SEQ-23 | SNP23 | 900277 | 41835 | rs 2518463 | T16C | AAAAGTTTTTCTCAGYGTGA |
| | | | | | | GTATTATGAGG |
| SEQ-24 | SNP24 | 900340 | 44295 | | C13A | GGGCCCTGGCATMAGTACTG |
| | | | | | | TTT |
| SEQ-25 | SNP25 | 900303 | 45354 | rs 2842936 | A16G | TAGCAGAGTAAAAATRTCAC |
| | | | | | | TCTGCTCGAGG |
| SEQ-26 | SNP26 | 900278 | 45429 | rs 2842935 | A16G | CCAACTGATCTTCAARGTTG |
| | | | | | | TCCTCTGTGAT |
| SEQ-27 | SNP27 | 900319 | 46390 | rs 2842934 | C16T | AGCATTAGTTGCCATYAATC |
| | | | | | | CAGGTGATCGC |
| SEQ-28 | SNP28 | 900311 | 46777 | rs 2859778 | T16G | TGGTCACTTGCGTATKCCAG |
| | | | | | | GTATTGTTCAA |
| SEQ-29 | SNP29 | 900312 | 47890 | rs 4712327 | T16C | TATAGCATGGAAATAYTGAA |
| | | | | | | TTACTTAGTTG |
| SEQ-30 | SNP30 | 900292 | 48260 | rs 2842955 | A16C | AACAGGTTAGGCTCCMCATC |
| | | | | | | AGTGAAATAAG |
| SEQ-31 | SNP31 | 900313 | 48568 | rs 2842952 | A16G | CTTTTTTTTCGAGAGRGAGT |
| | | | | | | TTCGCTCTTGT |
| SEQ-32 | SNP32 | 900304 | 49788 | rs 2518467 | G16C | CGTGCCCAGCCTTATSTTAG |
| | | | | | | TATTTTATATA |
| SEQ-33 | SNP33 | 900305 | 49921 | rs 2842951 | A16G | CTCCTTAGATTGTACRTTGTC |
| | | | | | | AAGTACTGAT |
| SEQ-34 | SNP34 | 900293 | 50426 | rs 2842950 | A16G | GTCTAGCCAGGCTCCRTAGA |
| | | | | | | AACTGGAGTGC |
| SEQ-35 | SNP35 | 900324 | 51526 | rs 6921269 | G16T | GGGAAAGAAGTTTCAKTATC |
| | | | | | | TCCTGTGTGTT |
| SEQ-36 | SNP36 | 900280 | 52782 | rs 2842947 | G16A | CTGGAGGTGGAGTCTRAGGA |
| | | | | | | TACTGCTCTTA |
| SEQ-37 | SNP37 | 900281 | 54592 | rs 1800584 | G16A | CTCTTTCTTGTTTCARGTAAA |
| | | | | | | ATATGCAATA |
| SEQ-38 | SNP38 | 900332 | 54648 | | T16G | TTTTGAAGAACGACAKAAAA |
| | | | | | | GTTGGGGAAT |
| SEQ-39 | SNP39 | 900283 | 55383 | rs 1802650 | A16T | GGCCTGACATTCTTTWTGAA |
| | | | | | | ATTTAGAAATG |
| SEQ-40 | SNP40 | 900284 | 56323 | rs 2842944 | C16G | GGTCTCACTTTGTTGSCCAC |
| | | | | | | GCTGATGTTGA |
| SEQ-41 | SNP41 | 900285 | 56945 | rs 7886 | A16T | CTTAGGTAGTTGATCWTTTA |
| | | | | | | TGTAATATGTG |

Reference SNPs:

| | | | | | | |
|---|---|---|---|---|---|---|
| SEQ-42 | SNP42 | 900326 | 36369 | | C11G | TGCTTTTCATSAGGAACAAGG |
| SEQ-43 | SNP43 | 900327 | 37528 | | G11A | TCCTCTTTGCTGAAAAGCGGT |
| SEQ-44 | SNP44 | 900276 | 41649 | rs 1800462 | G16C | ATTTTATGCAGGTTTSCAGA |
| | | | | | | CCGGGGACACA |
| SEQ-45 | SNP45 | 900328 | 41767 | | A11C | CCTGGAACCAMAGTATTTAA |
| | | | | | | GG |
| SEQ-46 | SNP46 | 900329 | 45684 | | G11A | TCATTGTACTRTTGCAGTATT |
| SEQ-47 | SNP47 | 900279 | 46376 | rs 1800460 | G16A | ATTTGGGATAGAGGARCATT |
| | | | | | | AGTTGCCATTA |
| SEQ-48 | SNP48 | 900330 | 46404 | | G11A | CCAGGTGATCRCAAATGGTA |
| | | | | | | A |
| SEQ-49 | SNP49 | 900331 | 54679 | | A11G | TCTTTTTGAARAGTTATATCT |
| SEQ-50 | SNP50 | 900282 | 54686 | rs 1142345 | A16G | TTTTTGAAAAGTTATRTCTA |
| | | | | | | CTTACAGAAAA |

TABLE 2

Primer and probe sequences of SNPs 1-50. The first column describes the
SNP-ID for SNPs 1-50, the second column describes the primer and probe
ID of each SNP. The nomenclature of primers and probes is as follows:
SP900xxx stands for the respective SNP (for example SP900295 is SNP 1)
followed by one or more alphabetic letters: F and R describe the forward
and reverse primers, or one of the four base symbols A, C, G, T followed
by a “+” or “−” describe the probes; the 5′ dye type of the probes are
symbolized by FAM, VIC, Tet. “+” probes have a MGB/DarkQuencher at 3′
end, “−” probes use TAMRA as Quencher. “Out” at the end of the primer's
name stands for first primers in nested PCR or outer primers. “AoD”
stands for Assays-on-Demand ™ from Applied Biosystems (commercially
available assays).

| | | | | | |
|---|---|---|---|---|---|
| SEQ-ID | SNP-ID | Primer/Probe-ID | Sequence | bp | Tm °C. |

| | | | | | |
|---|---|---|---|---|---|
| SEQ_51 | SNP1 | SP900295C + Fam | TATTTTTTCTcCTCCTTGCAT | 21 | 66.4 |
| SEQ_52 | SNP1 | SP900295F | TTCTCCAACCTGTTAGCAATCCTA | 24 | |
| SEQ_53 | SNP1 | SP900295R | GTGAAAGTGAATTATATGGATGATGGTAA | 29 | |
| SEQ_54 | SNP1 | SP900295T + Vic | AGTATTTTTTCTtCTCCTTGCA | 22 | 66.4 |
| SEQ_55 | SNP2 | SP900294A + Fam | TTTCTCTTTCAaATAACACTAT | 22 | 65.5 |
| SEQ_56 | SNP2 | SP900294F | CAACATAGCAACACCCTGTATCAAG | 25 | |
| 5EQ_57 | SNP2 | SP900294G + Vic | TCTCTTTCAgATAACACTAT | 20 | 65.2 |
| 5EQ_58 | SNP2 | SP900294R | CCCATAAAACAGGCTGTCAGAAG | 23 | |
| SEQ_59 | SNP3 | SP900296F | CTGGCCCTCTTTGTGTTTAAAAA | 23 | |
| SEQ_60 | SNP3 | SP900296G + Fam | TCTCGATgTTATTGAAC | 17 | 65.9 |
| SEQ_61 | SNP3 | SP900296R | CAGAGGAAAATATTCAATTAAGGGTTAAG | 29 | |
| SEQ_62 | SNP3 | SP900296T + Vic | TTTTCTCGATtTTATTGAAC | 20 | 66.2 |
| | SNP4 | AoD | C_1916835_10 | | |
| SEQ_63 | SNP5 | SP900297A − Fam | AGCCCGCACaGCCTTGCAG | 19 | 65.6 |
| SEQ_64 | SNP5 | SP900297F | TGTTCCCGGCCGATAGG | 17 | |
| SEQ_65 | SNP5 | SP900297g − Tet | CCCGCAGgGCCTTGCAG | 17 | 65.8 |
| SEQ_66 | SNP5 | SP900297R | GCTGTGCCAGAGAATTACTACAACA | 25 | |
| SEQ_67 | SNP6 | SP9002730 − Fam | TAGCCCGGGAATTcCCCCTTC | 21 | 65.9 |
| SEQ_68 | SNP6 | SP900273F2 | GGCAACATCGCGACGAA | 17 | |
| SEQ_69 | SNP6 | SP900273R2 | ATACCTCCTGCCCCGGATTA | 20 | |
| SEQ_70 | SNP6 | SP900273T − Tet | TAGCCCGGGAATTtCCCCTTCTT | 23 | 65.5 |
| SEQ_71 | SNP7 | SP900298A + Fam | ATTTCACAAaAATTTCT | 17 | 66.6 |
| SEQ_72 | SNP7 | SP900298F | GCACATTACAAGAATTAAGGAAGGG | 25 | |
| SEQ_73 | SNP7 | SP900298R | TTGAGGACTTTGTTTGTGGGC | 21 | |
| SEQ_74 | SNP7 | SP900298T + Vic | AAATTTCACAATAATTTCT | 19 | 66.5 |
| SEQ_75 | SNP8 | SP900338A + Fam | TCCTACAGCTaACTGAATA | 19 | 66.4 |
| SEQ_76 | SNP8 | SP900338F | CATGGGTACTTTCCTCCTTTCATAA | 25 | |
| SEQ_77 | SNP8 | SP900338R | TGAGGAAGGTGGCCAAATATACA | 23 | |
| SEQ_78 | SNP8 | SP900338T + Vic | TCCTACAGCTtACTGAATA | 19 | 66.4 |
| SEQ_79 | SNP9 | SP900314F | CTTATAATGTAGGGTGATGTGAGTGGAT | 28 | |
| SEQ_80 | SNP9 | SP900314g + Fam | AAACCTAGATTgATTTATTT | 20 | 66.2 |
| SEQ_81 | SNP9 | SP900314R | GCGAGACGCTGCCTCAAA | 18 | |
| SEQ_82 | SNP9 | SP900314T + Vic | AACCTAGATTtATTTATTTATTT | 24 | 65.8 |
| SEQ_83 | SNP10 | SP900315A + Fam | CACATTTAATTCTaCACATTT | 21 | 66.3 |
| SEQ_84 | SNP10 | SP900315F | TGTTCTATCAAAAAGTGACTTTGAGATAGA | 30 | |
| SEQ_85 | SNP10 | SP900315R | ATGCACTGTGAGTCGGGAGAC | 21 | |
| SEQ_86 | SNP10 | SP900315T + Vic | CACATTTAATTCTtCACATTT | 21 | 66.8 |
| SEQ_87 | SNP11 | SP900299A + Fam | TCTAACAaCCACTTTACT | 18 | 66 |
| SEQ_88 | SNP11 | SP900299F | CTGCCCAGAACAAGGAATGTC | 21 | |
| SEQ_89 | SNP11 | SP900299R | AGTAGTCTTCATAGCAGCAATAAATCATG | 29 | |
| SEQ_90 | SNP11 | SP900299T + Vic | TCTAACAtCCACTTTACT | 18 | 65.7 |
| SEQ_91 | SNP12 | SP900300A + Fam | CAAGCCTCaGAAGTA | 15 | 66.1 |
| SEQ_92 | SNP12 | SP900300F | TCAACATTAATTTCATGGTACGTTCTC | 27 | |
| SEQ_93 | SNP12 | SP900300g + Vic | CAAGCCTCgGAAGTA | 15 | 65.7 |
| SEQ_94 | SNP12 | SP900300R | GAAACTACAGGAGTTACACTTCTCAGGTT | 29 | |
| SEQ_95 | SNP13 | SP900301A + Fam | AAAGAATTTTCaAAACATC | 19 | 66.3 |
| SEQ_96 | SNP13 | SP900301F | TCCATGGCTCCAGAGGCTC | 19 | |
| SEQ_97 | SNP13 | SP900301R | CAGGGCTTTCCTGATTAGTAATTAAAAATA | 30 | |
| SEQ_98 | SNP13 | SP900301T + Vic | AAGAATTTTCtAAACATCC | 19 | 66.3 |
| SEQ_99 | SNP14 | SP900274A + Fam | CACCTACCAaACAAT | 15 | 66.7 |
| SEQ_100 | SNP14 | SP900274F | GTTGGGAATATTAAGTGAGATAATGAATGA | 30 | |
| SEQ_101 | SNP14 | SP900274R | AGTCCACTCTTGCCTTTAAGGAAA | 24 | |
| SEQ_102 | SNP14 | SP900274T + Vic | TTCACCTACCAtACAATT | 18 | 66.4 |
| SEQ_103 | SNP15 | SP900337C + Fam | CTTCAGGCTATcAAAGA | 17 | 66 |
| SEQ_104 | SNP15 | SP900337F | AATGAATGAAAAGTGTTCACCTACCA | 26 | |
| SEQ_105 | SNP15 | SP900337R | CATACCATTTCATCTCAACCGC | 22 | |
| SEQ_106 | SNP15 | SP900337T + Vic | TCTTCAGGCTATtAAAGA | 18 | 66.1 |
| SEQ_107 | SNP16 | SP900316C + Fam | ATTATCTTGcCTTAATGATGA | 21 | 66.6 |
| SEQ_108 | SNP16 | SP900316F | GCGGAAAAGCGGTTGAGAT | 19 | |
| SEQ_109 | SNP16 | SP900316R | CACATCCTGTTAAATCACCCAAAG | 24 | |
| SEQ_110 | SNP16 | SP900316T + Vic | ATTATCTTGtCTTAATGATGAAT | 23 | 67 |
| SEQ_111 | SNP17 | SP900336F | GGTGATTTAACAGGATGTGAGTTTTAAA | 28 | |
| SEQ_112 | SNP17 | SP900336G + Fam | CATTTTCAGgAAATACA | 17 | 66.5 |
| SEQ_113 | SNP17 | SP900336R | AAGACTTCATACCTGTTTCTGTTGTTTCT | 29 | |
| SEQ_114 | SNP17 | SP900336T + Vic | CCATTTTCAGtAAATACA | 18 | 66.6 |
| SEQ_115 | SNP18 | SP900317C + Fam | TTACATTTcCTGGATCCT | 18 | 66.1 |
| SEQ_116 | SNP18 | SP900317F | GAAGTCTTTCTGGATTGAGTTTTGAA | 26 | |
| SEQ_117 | SNP18 | SP900317R | CCACCTACAAAAACTGAACCACAT | 24 | |
| SEQ_118 | SNP18 | SP900317T + Vic | TACATTTtCTGGATCCTT | 18 | 66.4 |
| SEQ_119 | SNP19 | SP900318A + Fam | CTCTACAAAaAGAATTC | 17 | 66.7 |
| SEQ_120 | SNP19 | SP900318F | ACCAGTGATTAAGAAAGTATTTCTTGTGA | 29 | |
| SEQ_121 | SNP19 | SP900318g + Vic | TCTACAAAgAGAATTCA | 17 | 66.5 |
| SEQ_122 | SNP19 | SP900318R | GGGTAACTCATAGTAAAAGTGGCTTGTT | 28 | |
| SEQ_123 | SNP20 | SP900275A + Fam | ATGTAACAaGTGCCTTC | 17 | 66.5 |
| SEQ_124 | SNP20 | SP900275F | GCACAGTTATGATTTTATGTCAAGTGAA | 28 | |
| SEQ_125 | SNP20 | SP900275R | ATTTTTAGTGCGTGATTTAGCATAGTG | 27 | |
| SEQ_126 | SNP20 | SP900275T + Vic | ATGTAACAtGTGCCTTC | 17 | 67 |
| SEQ_127 | SNP21 | SP900335A + Fam | CTCTGTAAAAAATTaTTGTATCC | 23 | 66 |
| SEQ_128 | SNP21 | SP900335C + Vic | TCTGTAAAAAATTcTTGTATCC | 22 | 66 |
| SEQ_129 | SNP21 | SP900335F | GGGATATGGATACAATTATTTACCCAAA | 28 | |
| SEQ_130 | SNP21 | SP900335R | TGGTGTGGAAATCAGTGAACTTG | 23 | |
| SEQ_131 | SNP22 | SP900334C + Fam | TCACcGAAATTC | 12 | 65.9 |
| | SNP22 | SP900334F | =SP900328F | 11 | |
| | SNP22 | SP900334R | =SP900328R | 11 | |
| SEQ_132 | SNP22 | SP900334T + Vic | ACCAATCACtGAAATT | 16 | 66.2 |
| | SNP23 | AoD | C_396314_10 | | |
| SEQ_133 | SNP24 | SP900340A + Fam | CCTGGCATaAGTACTGT | 17 | 66.1 |
| SEQ_134 | SNP24 | SP900340C + Vic | CTGGCATcAGTACTGT | 16 | 66.1 |
| SEQ_135 | SNP24 | SP900340F | CCCCAGGCCAATTATATCAGAA | 22 | |
| SEQ_136 | SNP24 | SP900340R | AACTTTGCCTGCAGATTGGAA | 21 | |
| SEQ_137 | SNP25 | SP900303A + Fam | AGTAAAAATaTCACTCTGCTC | 21 | 65.8 |
| SEQ_138 | SNP25 | SP900303F | GATAATTGGTTGACCTGCAGATTTATC | 27 | |
| SEQ_139 | SNP25 | SP900303G + Vic | AGAGTAAAAATgTCACTCTG | 20 | 66.1 |
| SEQ_140 | SNP25 | SP900303R | GCTTGCTATAAAATTCTAACAATGTTTCC | 29 | |
| SEQ_141 | SNP26 | SP900278A + Fam | ATCTTCAAaGTTGTCCTC | 18 | 66 |
| SEQ_142 | SNP26 | SP900278F | CTCTGAAGTGAGTAACAGCCAACTG | 25 | |
| SEQ_143 | SNP26 | SP900278G + Vic | CTTCAAgGTTGTCCTC | 16 | 66.2 |
| SEQ_144 | SNP26 | SP900278R | GCACTTTATTGGCACCTTATTTTTTT | 26 | |
| SEQ_145 | SNP27 | SP900319C + Fam | TTAGTTGCCATcAATC | 16 | 66.9 |
| | SNP27 | SP900319F | =SP900279R | 14 | |
| | SNP27 | SP900319R | =SP900279Fout | 14 | |
| SEQ_146 | SNP27 | SP900319T + Vic | TTAGTTGCCATtAATCCA | 18 | 66.9 |
| SEQ_147 | SNP28 | SP900311F | CACAATCATCACCACCTCCACTA | 23 | |
| SEQ_148 | SNP28 | SP900311g − Fam | TCACTTGCCTATgCCAGGTATTGTTCA | 27 | 65.3 |
| SEQ_149 | SNP28 | SP900311R | CCCAGCCCACATAAAGTATTTTG | 23 | |
| SEQ_150 | SNP28 | SP90031IT − Tet | CTGGTCACTTGCCTATtCCAGGTATTGTT | 29 | 65 |
| SEQ_151 | SNP29 | SP900312A + Fam | CTAAGTAATTCAaTATTTCCATGC | 24 | 66.2 |
| SEQ_152 | SNP29 | SP900312F | CAAGTGATGAGTCTGCTCCATACAA | 25 | |
| SEQ_153 | SNP29 | SP900312g + Vic | CTAAGTAATTCAgTATTTCCAT | 22 | 66.2 |
| SEQ_154 | SNP29 | SP900312R | TGACCACATCTGTATACTCTTTCAATTAAA | 30 | |
| SEQ_155 | SNP30 | SP900292A + Fam | TAGGCTCCaCATCAG | 15 | 65.6 |
| SEQ_156 | SNP30 | SP900292C + Vic | TTAGGCTCCcCATCAG | 16 | 65.6 |
| SEQ_157 | SNP30 | SP900292F2 | GGGCAACGGAGTGAGATTTC | 20 | |
| SEQ_158 | SNP30 | SP900292R2 | ATTAGGTTTGGCAGTAAGCCTTACTG | 26 | |
| SEQ_159 | SNP31 | SP900313C + Fam | CGAAACTCcGTCTCG | 15 | 66.2 |
| SEQ_160 | SNP31 | SP900313F | CCAGCCTGGGCAACAAGA | 18 | |
| SEQ_161 | SNP31 | SP900313R | GCCAATATTTGTCCTACCAGAAAGA | 25 | |
| SEQ_162 | SNP31 | SP900313T + Vic | CGAAACTCtGTCTCGAA | 17 | 66.1 |
| SEQ_163 | SNP32 | SP900304C + Fam | AGCCTTATgTTAGTATTTT | 19 | 66.2 |
| SEQ_164 | SNP32 | SP900304F | CCAAAGTGCTGGGATTACAGATG | 23 | |
| SEQ_165 | SNP32 | SP900304g + Vic | CCCAGCCTTATcTTAGTAT | 19 | 66.6 |
| SEQ_166 | SNP32 | SP900304R | GTGCTAACATGGTAAGTACTGAGTACCA | 28 | |
| | SNP33 | AoD | C_396305_10 | | |
| SEQ_167 | SNP34 | SP900293C + Fam | CAGTTTCTAcGGAGCCT | 17 | 66.6 |
| SEQ_168 | SNP34 | SP900293F | TTCCCCACACTGAGGAAGGA | 20 | |
| SEQ_169 | SNP34 | SP900293R | GCACTTGCCTCCCCAACTT | 19 | |
| SEQ_170 | SNP34 | SP900293T + Vic | CCAGTTTCTAtGGAGCC | 17 | 66.6 |
| SEQ_171 | SNP35 | SP900324F | GCCTGTGTAGAGAAATGTAACAAATACC | 28 | |
| SEQ_172 | SNP35 | SP900324g + Fam | AAGTTTCAgTATCTCCTG | 18 | 66.4 |
| SEQ_173 | SNP35 | SP900324R | GGATGTTTAGTTGGATCATAAGAAAGAA | 28 | |
| SEQ_174 | SNP35 | SP900324T + Vic | AAGAAGTTTCAtTATCTCCT | 20 | 66.7 |
| SEQ_175 | SNP36 | SP900280C + Fam | AGTATCCTcAGACTCC | 16 | 67 |
| SEQ_176 | SNP36 | SP900280F | CTTCCGCCCCCTTCTAAGAG | 20 | |
| SEQ_177 | SNP36 | SP900280R | AAAGAACCTTTGGGAAGAAAATACAG | 26 | |
| SEQ_178 | SNP36 | SP900280T + Vic | CAGTATCCTtAGACTCC | 17 | 66.6 |
| SEQ_179 | SNP37 | SP900281A + Fam | TCTTGTTTCAaGTAAAATA | 19 | 66.5 |
| SEQ_180 | SNP37 | SP900281F | CCTGATGTCATTCTTCATAGTATTTTAACA | 30 | |
| SEQ_181 | SNP37 | SP900281G + Vic | TCTTGTTTCAgGTAAAAT | 18 | 66.1 |
| SEQ_182 | SNP37 | SP900281R | CCTTCTCAAGACAACGTATATTGCA | 25 | |
| SEQ_183 | SNP38 | SP900332A + Fam | CCAACTTTTaTGTCGTTCT | 19 | 65.9 |
| SEQ_184 | SNP38 | SP900332C + Vic | CAACTTTTcTGTCGTTCT | 18 | 65.5 |
| SEQ_185 | SNP38 | SP900332F | CATGTCAGTGTGATTTTATTTTATCTATGTCTC | 33 | |
| SEQ_186 | SNP38 | SP900332R | CCTGATGTCATTCTTCATAGTATTTTAACA | 30 | |
| SEQ_187 | SNP39 | SP900283A + Fam | TTCTAAATTTCAaAAAGAATGT | 22 | 65.8 |
| SEQ_188 | SNP39 | SP900283F | GACCACCTTGAACCCTACTGAAA | 23 | |
| SEQ_189 | SNP39 | SP900283R | AGGCGTGAGCCACTGCA | 17 | |
| SEQ_190 | SNP39 | SP900283T + Vic | ATTCTAAATTTCAtAAAGAATGT | 23 | 65.8 |
| SEQ_191 | SNP40 | SP900284c − Fam | TCTCACTTTGTTGcCCACGCTGAT | 24 | 65.8 |
| SEQ_192 | SNP40 | SP900284F | GGACCAACACAATTCTCTCCAGA | 23 | |
| SEQ_193 | SNP40 | SP900284g − Tet | TCTCACTTTGTTGgCCACGCTGAT | 24 | 65.8 |
| SEQ_194 | SNP40 | SP900284R | GGAGGACTGCTTGAGGCCTC | 20 | |
| | SNP41 | AoD | C_12091548_10 | | |
| SEQ_195 | SNP42 | SP900326C + Fam | TCCTcATGAAAAGC | 14 | 66.5 |
| SEQ_196 | SNP42 | SP900326F | CAAAGTCACTTTTTGATAGAACATTTCTC | 29 | |
| SEQ_197 | SNP42 | SP900326g + Vic | TCCTgATGAAAAGC | 14 | 66.5 |
| SEQ_198 | SNP42 | SP900326R | AAGTGGGTGAACGGCAAGAC | 20 | |
| SEQ_199 | SNP43 | SP900327C − Fam | CAACCGCTTTTCcGCAAAGAGG | 22 | 65.7 |
| SEQ_200 | SNP43 | SP900327F | TTCTGTTAATGTTTATCTGCTCATACCA | 28 | |
| SEQ_201 | SNP43 | SP900327R | GCAAGAGTGGACTGAGGGTATTTT | 24 | |
| SEQ_202 | SNP43 | SP900327T − Tet | TCAACCGCTTTTCtGCAAAGAGGAA | 25 | 65.8 |
| SEQ_203 | SNP44 | SP900276C − Fam | TCCCCGGTCTGcAAACCTGC | 20 | 66.3 |
| SEQ_204 | SNP44 | SP900276F | TCACTGATTTCCAGACCAACTACA | 24 | |
| SEQ_205 | SNP44 | SP900276G − Tet | CCCCGGTCTGgAAACCTGCA | 20 | 66.1 |
| SEQ_206 | SNP44 | SP900276R | TGTTCTTTGAAACCCTATGAACCTG | 25 | |
| SEQ_207 | SNP45 | SP900328A + Fam | CTGGAACCAaAGTATT | 16 | 66.4 |
| SEQ_208 | SNP45 | SP900328C + Vic | GTGGAACCAcAGTATT | 16 | 66.5 |
| SEQ_209 | SNP45 | SP900328F | ACAGAGCAGAATCTTTCTTACTCAGAAG | 28 | |
| SEQ_210 | SNP45 | SP900328R | GGGATATGGATACAATTATTTACCCAAA | 28 | |
| SEQ_211 | SNP46 | SP900329C + Fam | TACTGCAAcAGTACAATG | 18 | 66.2 |
| SEQ_212 | SNP46 | SP900329F | TCAACCTACCTGGGAAGATCAAA | 23 | |
| SEQ_213 | SNP46 | SP900329R | GGCCCTCTTTCCTTGACTATTCA | 23 | |
| SEQ_214 | SNP46 | SP900329T + Vic | ATACTGCAAtAGTACAATGA | 20 | 66.4 |
| SEQ_215 | SNP47 | SP900279C + Fam | CAACTAATGcTCCTCTAT | 18 | 66.5 |
| SEQ_216 | SNP47 | SP900279Fout | GCTAAACAAAAAAAGAAAAATTACTTACCAT | 31 | |
| SEQ_217 | SNP47 | SP900279F | TGCGATCACCTGGATTGATG | 20 | |
| SEQ_218 | SNP47 | SP900279Rout | TCTTAAAGATTTGATTTTTCTCCCATAAA | 29 | |
| SEQ_219 | SNP47 | SP900279R | TTCTGGTAGGACAAATATTGGCAA | 24 | |
| SEQ_220 | SNP47 | SP900279T + Vic | CAACTAATGtTCCTCTATC | 19 | 66.9 |
| SEQ_221 | SNP48 | SP900330A + Fam | ATCCAGGTGATCaCAAA | 17 | 66.1 |
| SEQ_222 | SNP48 | SP900330F | TTCTGGTAGGACAAATATTGGCAA | 14 | |
| SEQ_223 | SNP48 | SP900330g + Vic | CCAGGTGATCgCAAA | 15 | 66.2 |
| SEQ_224 | SNP48 | SP900330R | GCTAAACAAAAAAAGAAAAATTACTTACCAT | 14 | |
| SEQ_225 | SNP49 | SP900331C + Fam | TAACTcTTCAAAAAGAC | 17 | 66 |
| SEQ_226 | SNP49 | SP900331F | CATGTCAGTGTGATTTTATTTTATCTATGTCTC | 33 | |
| SEQ_227 | SNP49 | SP900331R | GAGAAGGTTGATGCTTTTGAAGAAC | 25 | |
| SEQ_228 | SNP49 | SP900331T + Vic | ATAACTtTTCAAAAAGAC | 18 | 65.7 |
| SEQ_229 | SNP50 | SP900282A + Fam | TTTGAAAAGTTATaTCTACTTACA | 24 | 65.1 |
| SEQ_230 | SNP50 | SP900282F | TGATGCTTTTGAAGAACGACATAAA | 25 | |
| SEQ_231 | SNP50 | SP9002820 + Vic | TTTTTGAAAAGTTATgTCTACTTA | 24 | 65.3 |
| SEQ_232 | SNP50 | SP900282R | TCCTCAAAAACATGTCAGTGTGATT | 25 | |

TABLE 3
Example of a TaqMan PCR Protocol

| TaqMan PCR Protocol | | Experiment # | SSPif031028A |
|---|---|---|---|
| Primer #1 | SP900282F | Probe #1 | SP900282A + Fam |
| Primer #2 | SP900282R | Probe #2 | SP900282g + Vic |
| DNA plate | MDA 3 | Primer #1 | 100 μM |
| DNA plate | MDA 10 | Primer #2 | 100 μM |
| DNA plate | MDA 11 | Probe #1 | 50 μM |
| DNA plate | MDA 12 | Probe #2 | 50 μM |
| Quencher | MGB/non | | |
| | fluorescent | | |

| | | |
|---|---|---|
| PCR machine | Biometra | Number of samples |
| Taq | qPCR Mastermix | 440 |
| Polymerase | Fa. Eurogentec | |

| | | | | Mastermix |
|---|---|---|---|---|
| Reaction vol. | [μL] | Endkonz. | | [μL] |
| H2O | 3.318 | | | 1460 |
| TQMMM | 3.5 | 1x | | 1540 |
| Primer #1 | 0.063 | 0.9 | μM | 28 |
| Primer #2 | 0.063 | 0.9 | μM | 28 |
| Probe #1 | 0.028 | 0.2 | μM | 12.3 |
| Probe #2 | 0.028 | 0.2 | μM | 12.3 |
| Template DNA | 3 | 2-20 | ng | at 80° C. 30′ dried down |

| | | | |
|---|---|---|---|
| Reaction vol. | 7 | each | 3 μL Template (dried) |
| | | and each | 7 μL Mastermix |

| | Temp | | | Back | Number |
|---|---|---|---|---|---|
| PCR Program | [° C.] | Time | Step | to step | of cycles |
| Pre-incubation | 95 | 10′ | 1 | | |
| Denaturing | 94 | 15″ | 2 | | |
| Primer annealing | 61 | 1′ | 3 | 2 | 54 |
| Hold | 8 | 8′ | 4 | | |

### TPMT Assay

Erythrocyte lysates were analyzed for TPMT activity by a HPLC method using 6-thioguanine as substrate described in Kroeplin, T. et al., Eur. J. Clin. Pharmacol (1998) 54: 265-271.

### Sequencing of VNTR

Sequencing of the VNTR of the TPMT gene was performed with an ABI Prism™ 3700 (Applied Biosystems) using a protocol as described by the manufacturer with the following primers:

| | |
|---|---|
| VNTR-Seq1 | gctccgccctgcccattt (forward) |
| and | |
| VNTR-Seq2 | gtcattggtggcggaggc (reverse) |

In general, molecular techniques were performed according to Sambrook et al. Molecular Cloning, A Laboratory Manual, 3^{rd} Ed. 2000, Cold Spring Harbor Laboratory Press.

The VNTR regions that were amplified with the primers VNTR-Seq1+2 ranged in length from 233 to 377 bp with 1-6 repeats of A (gtcattggtggcggaggc), 1-3 repeats of B (gaggcggggcgcgggcg), and 1 repeat of C (gaggcggggcgcggaga).

### Results

From the ca. 1300 DNA samples we identified 135 unique haplotypes in the TPMT gene. Table 4a shows the allele frequencies of all polymorphic SNPs; 20 SNPs were found to be monomorphic in our 1300 DNA samples (listed in Table 4b). Surprisingly, 5 out of 9 reference SNPs were monomorphic in the tested population. Although 5 out of 6 SNPs were taken from one patent application as reference SNPs and were meant to be used as benchmark SNPs!

TABLE 4a
Allele frequencies of all 30 polymorphic SNPs in ca.1300
samples. Reference SNPs are marked with a “R” in the
second column, linked SNPs are shaded, other SNPs, which are
mentioned particularly in the text are marked either with a
comma (,) or a dash (-).

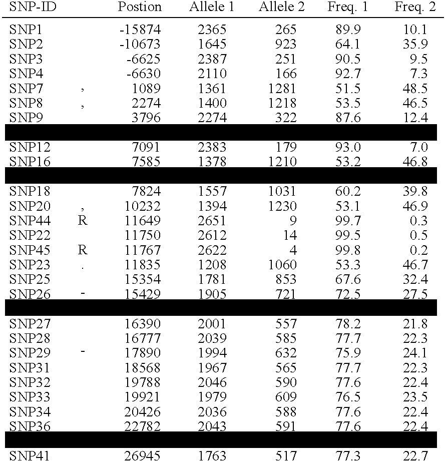

TABLE 4b
20 monomorphic SNPs were found in the 1300 DNA samples tested.

| SNP-ID | Bay-SNP-ID | |
|---|---|---|
| SNP5 | 900297 | monomorphic |
| SNP6 | 900273 | monomorphic |
| SNP11 | 900299 | monomorphic |
| SNP13 | 900301 | monomorphic |
| SNP14 | 900274 | monomorphic |
| SNP15 | 900337 | monomorphic |
| SNP19 | 900318 | monomorphic |
| SNP21 | 900335 | monomorphic |
| SNP24 | 900340 | monomorphic |
| SNP30 | 900292 | monomorphic |
| SNP35 | 900324 | monomorphic |
| SNP37 | 900281 | monomorphic |
| SNP38 | 900332 | monomorphic |
| SNP39 | 900283 | monomorphic |
| SNP40 | 900284 | monomorphic |

Reference SNPs:

| | | |
|---|---|---|
| SNP42 | 900326 | monomorphic |
| SNP43 | 900327 | monomorphic |
| SNP46 | 900329 | monomorphic |
| SNP48 | 900330 | monomorphic |
| SNP49 | 900331 | monomorphic |

Table 5 shows all different haplotypes of 30 polymorphic SNPs in 5′ to 3′ direction on the TPMT gene (from left to right in the table). Positions of SNPs are mentioned in reference to the accession number AL589723 (reverse complement). To get a better overview, the wild type genotype is symbolized in Table 5 with a comma (,), the heterocygote is marked with an (o) and the mutant homocygote is marked with an (X). The real genotypes can be read from the bottom of the table. It can be seen from the table that between SNP 47 and SNP 27 starts a transition of one haplotype block to another one, representing probably a crossover point of maternal and paternal chromosomes in meiosis. The downstream part of the TPMT gene, which starts in Table 5 with SNP 27, codes for the last four exons of the TPMT protein. This haplotype block contains SNPs that have in nearly all patients measured a similar allele frequency with very similar occurrences of wild type, heterocygote and mutant genotypes.

TABLE 5
All different haplotypes of 30 polymorphic SNPs in 5′ to 3′ direction on the TPMT gene (from left to right in the table). Reference SNPs are
marked with a “R” , linked SNPs are shaded, other SNPs, which are mentioned in the text are marked with a dash (-).

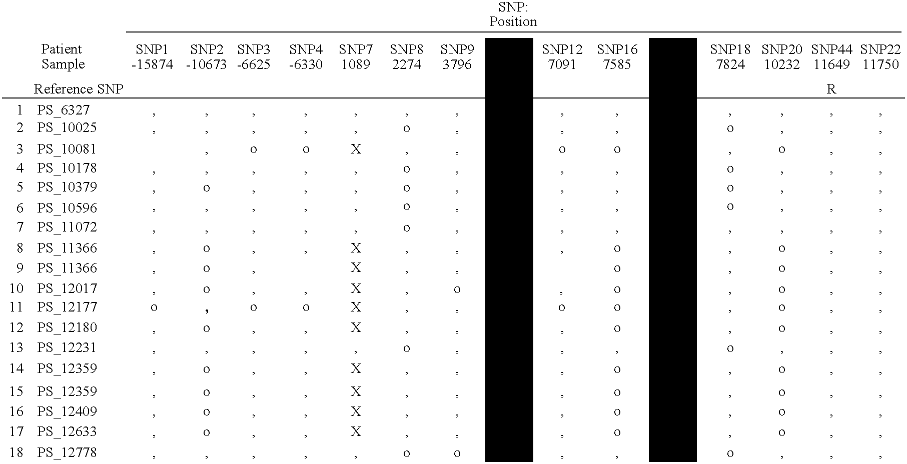

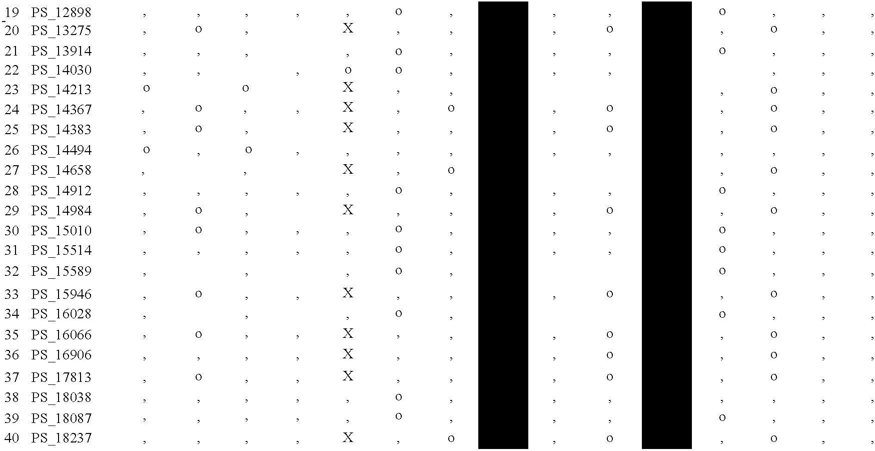

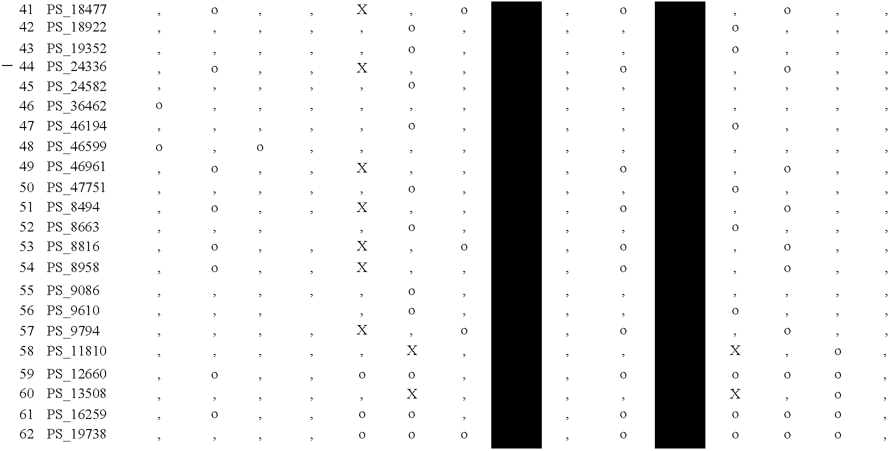

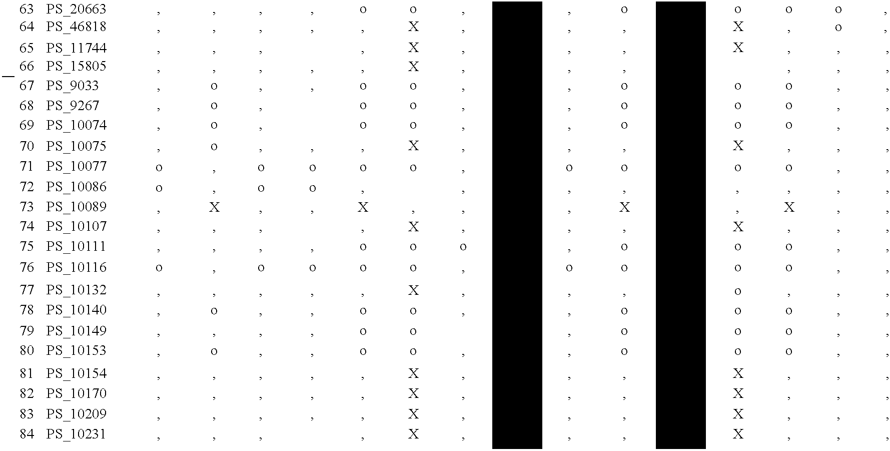

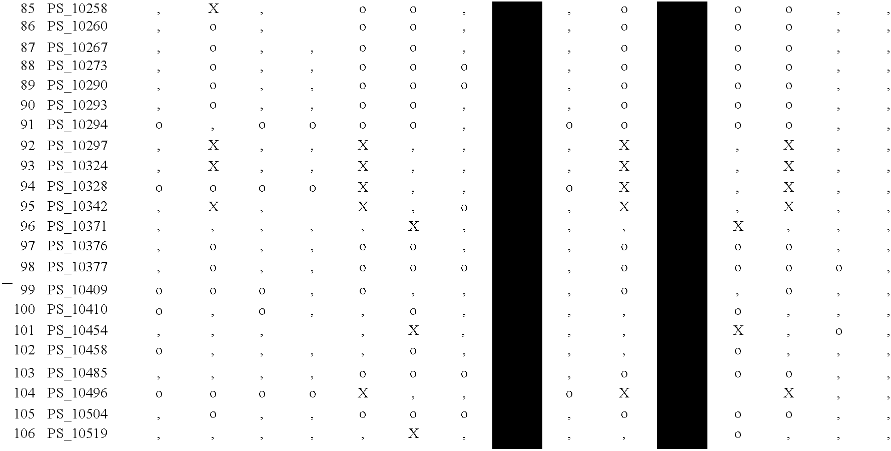

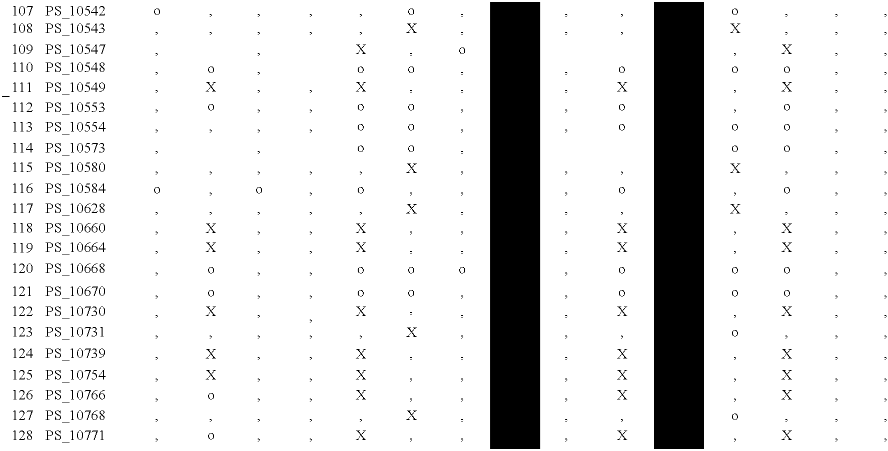

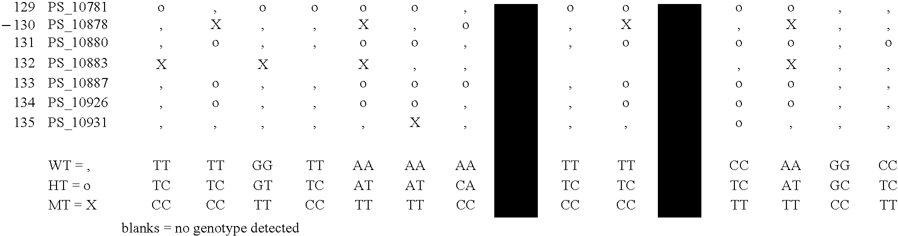

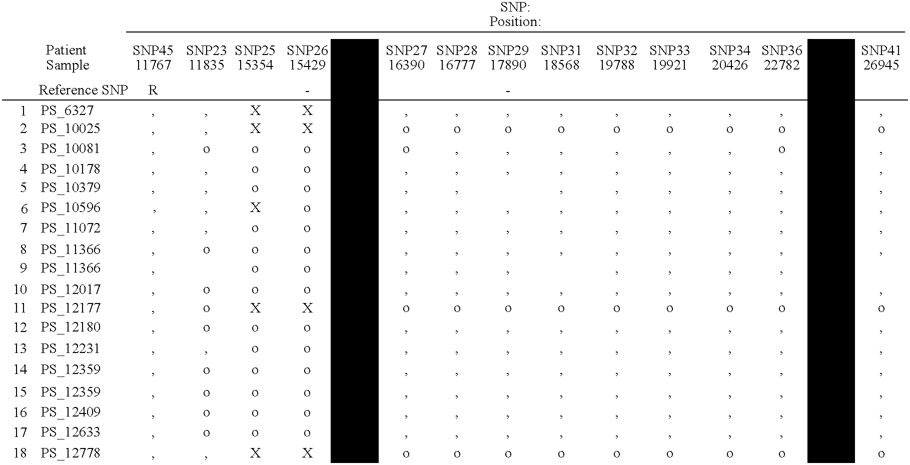

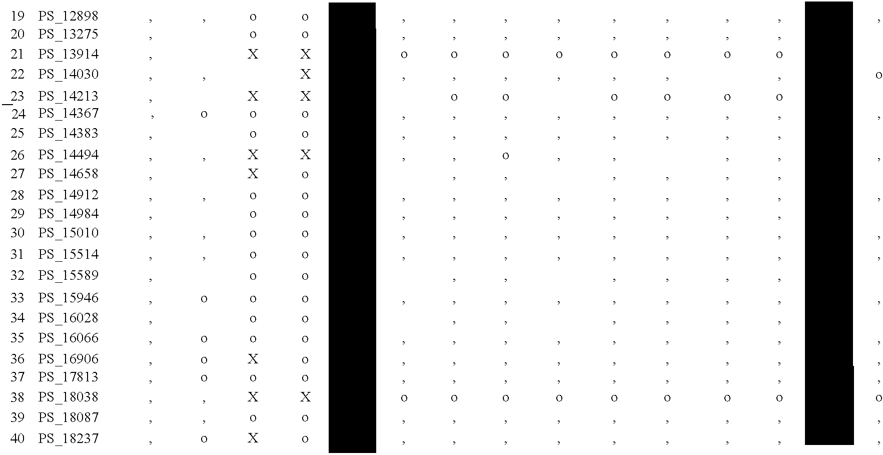

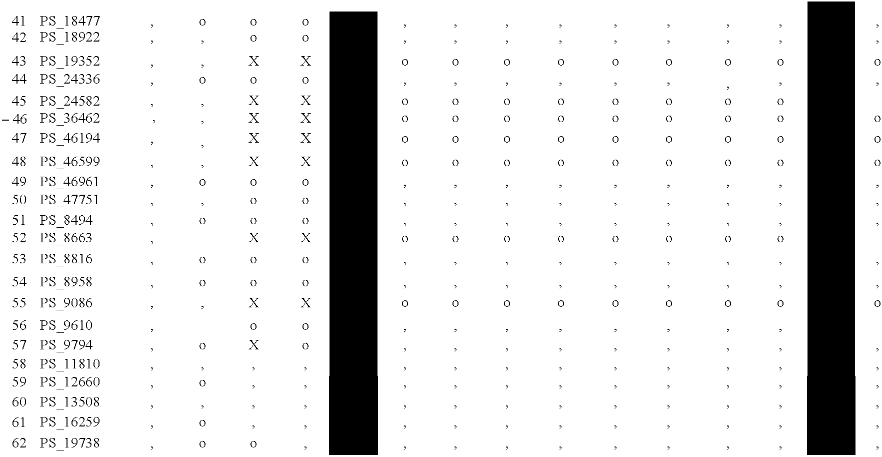

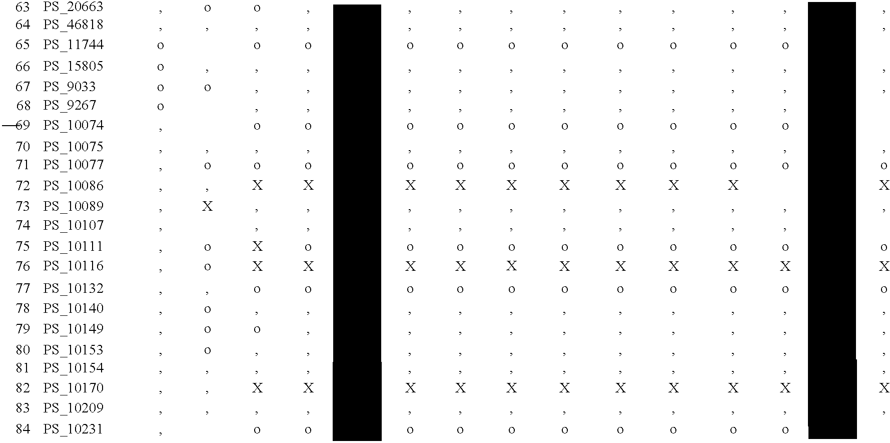

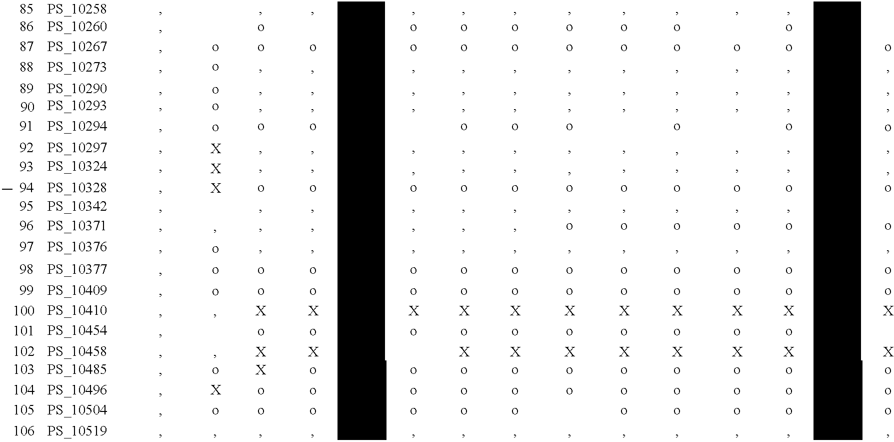

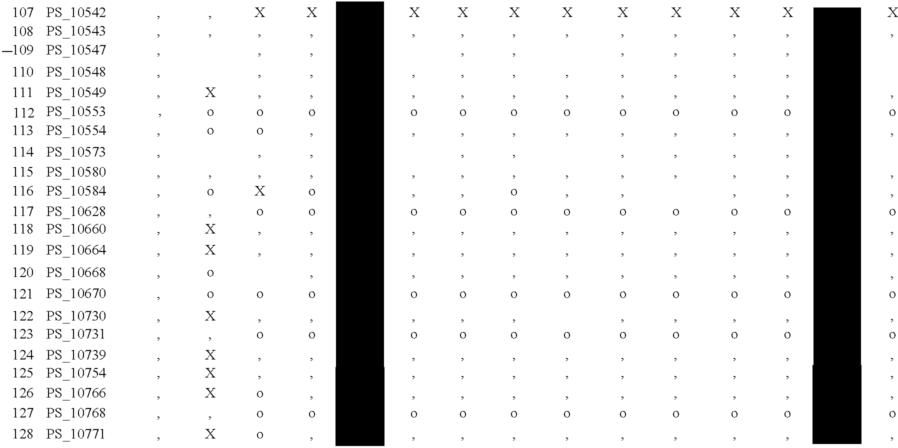

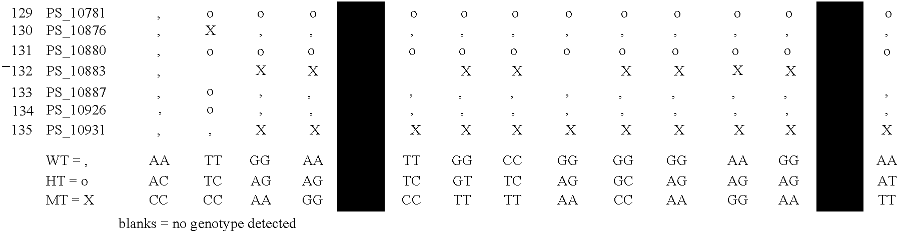

One exception in this haplotype block is SNP 50, which shows an independent pattern. The upstream part of the gene contains SNPs that show from patient to patient a more independent pattern of allele frequencies (probably a hot spot of recombination). Due to these two adjacent haplotype blocks within one gene it is a priori not possible to conclude linked SNPs merely from the fact that they are neighbors on a gene. But surprisingly we found that SNPs 10, 17, 47 and 50 are linked to each other, more precisely SNP 10 is highly linked to SNP 50 and SNP 17 is highly linked to SNP 47. Even more surprisingly we found that SNP 26 and 29 represent one haplotype that is linked to the reference SNPs 47 and 50 and to the SNPs 10 and 17 in the following way:

When SNP 26 being HT and SNP 29 being WT the TPMT enzyme is deficient.

When SNP 26 being MT and SNP 29 being WT the TPMT enzyme is more deficient.

When SNP 26 being MT and SNP 29 being HT the TPMT enzyme is deficient.

In a similar way one can identify from Table 5 other haplotypes that are linked to deficient TPMT enzyme activity:

When SNP 7 being MT and SNP 20 being HT the TPMT enzyme is deficient.

When SNP 7 being WT and SNP 8 being HT and SNP 20 being WT the TPMT enzyme is deficient.

In Table 5 other haplotypes can be identified to describe TPMT deficient individuals. For example any of the haplotypes from row 1 to 57 in Table 5 can be used to describe individuals who are TPMT enzyme deficient using two or up to all of the following SNPs: SNP1, 2, 3, 4, 7, 8, 9, 10, 12, 16, 17, 18, 20, 22, 23, 25, 26, 27, 28, 29, 31, 32, 33, 34, 36, 41. In Table 6 is an example of 10 individuals with their respective haplotypes from Table 5:

TABLE 6
Examples of haplotypes of TPMT deficient individuals

| | SNP1 | SNP2 | SNP3 | SNP4 | SNP7 | SNP8 | SNP9 | SNP10 | SNP12 | SNP16 | SNP17 | SNP18 | SNP20 | SNP22 |
|---|---|---|---|---|---|---|---|---|---|---|---|---|---|---|
| 1 | wt | wt | wt | wt | wt | wt | wt | MT | wt | wt | MT | wt | wt | wt |
| 2 | wt | wt | wt | wt | wt | ht | wt | ht | wt | wt | ht | ht | wt | wt |
| 3 | wt | wt | wt | wt | wt | ht | wt | ht | wt | wt | ht | ht | wt | wt |
| 4 | wt | wt | wt | wt | wt | ht | wt | ht | wt | wt | ht | ht | wt | wt |
| 5 | wt | wt | wt | wt | wt | ht | wt | ht | wt | wt | ht | wt | wt | wt |
| 6 | wt | ht | wt | wt | MT | wt | ht | ht | wt | ht | ht | wt | ht | wt |
| 7 | ht | wt | ht | ht | MT | wt | wt | ht | ht | ht | ht | wt | ht | wt |
| 8 | wt | wt | wt | wt | wt | ht | wt | ht | wt | wt | ht | ht | wt | wt |
| 9 | wt | ht | wt | wt | MT | wt | wt | ht | wt | ht | ht | wt | ht | wt |
| 10 | wt | wt | wt | wt | wt | ht | ht | ht | wt | wt | ht | ht | wt | wt |
| WT | TT | TT | GG | TT | AA | AA | AA | TT | TT | TT | GG | CC | AA | CC |
| HT | TC | TC | GT | TC | AT | AT | CA | AT | TC | CT | GT | TC | TA | TC |
| MT | CC | CC | TT | CC | TT | TT | CC | AA | CC | CC | TT | TT | TT | TT |

| | SNP23 | SNP25 | SNP26 | SNP27 | SNP28 | SNP29 | SNP31 | SNP32 | SNP33 | SNP34 | SNP36 | SNP41 |
|---|---|---|---|---|---|---|---|---|---|---|---|---|
| 1 | wt | MT | MT | wt | wt | wt | wt | wt | wt | wt | wt | wt |
| 2 | wt | MT | MT | ht | ht | ht | ht | ht | ht | ht | ht | ht |
| 3 | wt | ht | ht | wt | wt | wt | wt | wt | wt | wt | wt | wt |
| 4 | wt | MT | ht | wt | wt | wt | wt | wt | wt | wt | wt | wt |
| 5 | wt | ht | ht | wt | wt | wt | wt | wt | wt | wt | wt | wt |
| 6 | ht | ht | ht | wt | wt | wt | wt | wt | wt | wt | wt | wt |
| 7 | ht | MT | MT | ht | ht | ht | ht | ht | ht | ht | ht | ht |
| 8 | wt | ht | ht | wt | wt | wt | wt | wt | wt | wt | wt | wt |
| 9 | ht | ht | ht | wt | wt | wt | wt | wt | wt | wt | wt | wt |
| 10 | wt | MT | MT | ht | ht | ht | ht | ht | ht | ht | ht | ht |
| WT | TT | GG | AA | TT | GG | CC | GG | GG | GG | AA | GG | AA |
| HT | TC | AG | AG | TC | GT | TC | AG | GC | AG | AG | AG | AT |
| MT | CC | AA | GG | CC | TT | TT | AA | CC | AA | GG | AA | TT |

Each SNP in one row has to be combined with another one from the same row, whereas combinations can be two, three, four or up to all SNPs. In most cases it will be sufficient to take one or two of the SNPs 27, 28, 29, 31, 32, 33, 34, 36, 41 because they are very tightly linked to each other. (See complete Table 5).

A further example is given in Table 7 that shows the correlation of TPMT enzyme activity measured in healthy volunteers together with their individual haplotype of 10 SNPs. Erythrocyte lysates were analyzed for TPMT activity by a HPLC method using 6-thioguanine as substrate. The method is described in Kroeplin, T. et al., Eur. J. Clin. Pharmacol (1998) 54: 265-271. The enzyme activity was measured in nmol/gHb/h. The TPMT activity showed a range from 0 nmol/gHb/h to 106 nmol/gHb/h with a median of 46.6 nmol/gHb/h and a mean of 47.6. When setting the cutoff to 34.5 mmol/gHb/h the here presented haplotypes of patients whose TPMT value is below this cutoff have a sensitivity and specificity of 93% respectively. With this example, the responding haplotypes are further examples of haplotypes that constitute the different TPMT phenotypes in humans and can be used as an aid for therapy decision when respective patients have to be treated with thiopurines or derivatives.

TABLE 7
Correlation of SNPs and Haplotypes to TPMT Enzyme Activity

| | TPMT | | | | | | | | | | |
|---|---|---|---|---|---|---|---|---|---|---|---|
| | Enzyme | | | | | | | | | | |
| | Activity | SNP20 | SNP8 | | SNP26 | SNP29 | SNP10 | SNP17 | SNP44 | SNP47 | SNP50 |
| Patient | nmol/gHb/h | | Hap 2 | SNP7 | Hap 1 | | Hap 3 | | Reference SNPs | | |
| 976 | 0 | TT | TT | TT | GG | AA | TT | GG | CG | CC | AA |
| 979 | 0 | AA | TT | AA | AA | AA | AA | TT | CG | TT | GG |
| 1 | 13.7 | AA | AA | AA | AA | AA | AA | TT | GG | TT | GG |
| 6 | 17.0 | AT | TT | AA | AG | AA | AT | GT | GG | CT | AG |
| 7 | 17.2 | AT | TT | TT | GG | AG | AT | GT | GG | CT | AA |
| 9 | 18.7 | AA | TT | AA | GG | AG | AT | GT | GG | CT | AG |
| 16 | 20.3 | AT | TT | TT | AG | AA | AT | GT | GG | CT | AA |
| 20 | 21.0 | AT | TT | TT | AG | AA | AT | GT | GG | CT | AG |
| 22 | 21.2 | AT | TT | TT | GG | AG | AT | GT | GG | CT | AG |
| 23 | 21.6 | AT | TT | TT | GG | AG | AT | GT | GG | CT | AA |
| 24 | 21.7 | AA | AT | AA | GG | AG | AT | GT | GG | CT | AG |
| 25 | 21.7 | AA | AT | TT | AG | AA | NN | GT | GG | CT | AG |
| 26 | 21.7 | AA | AT | AA | AG | AA | AT | GT | GG | CT | AG |
| 28 | 22.4 | TT | AA | AA | AA | AA | AA | TT | NN | TT | GG |
| 30 | 23.0 | TT | AT | AA | AG | AA | AT | GT | GG | CT | AG |
| 32 | 23.4 | AT | TT | AA | AG | AA | AT | GT | GG | TT | AA |
| 33 | 23.7 | AA | AA | AA | AA | AA | AA | TT | GG | TT | GG |
| 34 | 23.9 | AT | TT | TT | AG | AA | AT | GT | GG | CT | AG |
| 977 | 24.0 | AT | TT | AA | AG | AA | AT | GT | GG | CT | AG |
| 36 | 25.0 | AT | TT | TT | AG | AA | AT | GT | GG | CT | AA |
| 37 | 25.0 | AT | TT | TT | GG | AG | AT | GT | GG | CT | AG |
| 38 | 25.1 | AA | AT | AA | AG | AA | AT | GT | GG | CT | AG |
| 41 | 25.7 | TT | AT | AA | GG | AG | AT | GT | NN | TT | AG |
| 42 | 25.8 | AT | TT | AT | AG | AA | AT | GT | GG | CT | AA |
| 43 | 26.0 | AA | AT | AA | AG | AG | AA | TT | GG | TT | GG |
| 44 | 26.1 | AT | TT | AA | AG | AA | AT | GT | GG | CT | AA |
| 45 | 26.3 | AA | AT | AA | AG | AA | AT | GT | GG | CT | AG |
| 46 | 26.5 | AA | AT | AA | AG | AA | AT | GT | GG | TT | AG |
| 50 | 27.5 | TT | TT | TT | AA | AA | AA | TT | GG | TT | GG |
| 51 | 27.6 | AA | AT | AA | AG | AA | AT | GT | GG | CT | AG |
| 52 | 27.6 | AT | AT | AT | AA | AA | AA | TT | GG | TT | GG |
| 59 | 28.9 | AT | TT | AA | AG | AA | AT | GT | GG | CT | AG |
| 60 | 29.2 | AT | AT | AT | AA | AA | AA | TT | GG | TT | GG |
| 61 | 29.2 | AT | TT | TT | GG | AG | AT | GT | GG | CT | AG |
| 63 | 29.4 | AT | AT | AT | AG | AG | AA | TT | GG | TT | GG |
| 65 | 29.8 | AA | AT | AA | GG | GG | AA | TT | GG | TT | GG |
| 66 | 29.8 | AT | TT | AT | AG | AG | AA | TT | GG | TT | GG |
| 68 | 30.3 | AT | AT | AT | AG | AG | AA | TT | GG | TT | GG |
| 70 | 30.4 | AA | AT | AA | AG | AA | AT | GT | GG | CT | AG |
| 71 | 30.6 | TT | TT | TT | AA | AA | AA | TT | GG | TT | GG |
| 74 | 31.2 | TT | TT | AA | AA | AA | AA | TT | GG | TT | GG |
| 75 | 31.2 | AA | AA | AA | AG | AG | AA | TT | GG | TT | GG |
| 77 | 31.6 | AA | AA | AA | AG | AG | AA | TT | GG | TT | GG |
| 78 | 31.7 | AT | AT | AT | AA | AA | AA | TT | GG | TT | GG |
| 79 | 31.7 | AT | AT | AT | AG | AA | AA | TT | GG | TT | GG |
| 81 | 31.9 | TT | TT | TT | AG | AG | AA | TT | GG | TT | GG |
| 83 | 32.1 | AT | AT | AT | AG | AG | AA | TT | GG | TT | GG |
| 87 | 32.4 | AA | AT | AA | AG | AG | AA | TT | GG | TT | GG |
| 92 | 32.8 | AT | AT | AT | AG | AA | AA | TT | GG | TT | GG |
| 93 | 32.8 | TT | TT | TT | AA | AA | AA | TT | GG | TT | GG |
| 95 | 32.8 | AA | AA | AA | GG | GG | AA | TT | GG | TT | GG |
| 96 | 32.8 | AA | AT | AA | GG | GG | AA | TT | GG | TT | GG |
| 97 | 33.1 | AT | TT | TT | AG | AA | AT | GT | GG | CT | AG |
| 98 | 33.1 | TT | TT | AA | AG | AG | AA | TT | GG | TT | GG |
| 100 | 33.1 | TT | TT | AA | AA | AA | AA | TT | GG | TT | GG |
| 102 | 33.4 | AT | AT | AT | AG | AG | AA | TT | GG | TT | GG |
| 104 | 33.6 | AT | AT | AA | AG | AG | AA | TT | GG | TT | GG |
| 105 | 33.6 | AA | AT | AA | AG | AA | AT | GT | GG | CT | AG |
| 107 | 33.7 | AA | AT | AT | AA | AA | AA | TT | GG | TT | GG |
| 108 | 33.8 | TT | TT | TT | AA | AA | AA | TT | GG | TT | GG |
| 114 | 34.2 | AT | TT | TT | GG | AG | AT | GT | GG | TT | AG |
| 115 | 34.2 | TT | AT | AA | AG | AG | AA | TT | GG | TT | GG |
| 119 | 34.5 | AT | AT | AT | AG | AG | AA | TT | GG | TT | GG |
| 122 | 34.6 | TT | TT | AA | AA | AA | AA | TT | GG | TT | GG |
| 135 | 35.5 | AA | AA | AA | AG | AG | AA | TT | GG | TT | GG |
| 136 | 35.6 | AT | AT | AT | AA | AA | AA | TT | GG | TT | GG |
| 139 | 35.8 | AT | AT | AT | AG | AG | AA | TT | GG | TT | GG |
| 141 | 35.9 | TT | TT | TT | AA | AA | AA | TT | GG | TT | GG |
| 142 | 36.0 | TT | TT | TT | AG | AG | AA | TT | GG | TT | GG |
| 152 | 36.7 | AT | AT | AT | GG | GG | AA | TT | GG | TT | GG |
| 154 | 37.0 | AA | AA | AA | AA | AA | AA | TT | GG | TT | GG |
| 155 | 37.0 | AT | AT | AT | AA | AA | AA | TT | GG | TT | GG |
| 156 | 37.0 | AA | AT | AA | AG | AG | AA | TT | GG | TT | GG |
| 158 | 37.2 | AT | AT | AT | AG | AG | AA | TT | GG | TT | GG |
| 165 | 37.5 | AA | AA | AA | AG | AG | AA | TT | GG | TT | GG |
| 170 | 37.7 | AT | TT | AT | AG | AG | AA | TT | GG | TT | GG |
| 171 | 37.8 | AT | AT | AT | AA | AA | AA | TT | GG | TT | GG |
| 172 | 37.8 | AT | TT | AT | GG | GG | AA | TT | GG | TT | GG |
| 174 | 37.9 | TT | TT | TT | GG | GG | AA | TT | GG | TT | GG |
| 180 | 38.2 | AA | AA | AA | AA | AA | AA | TT | GG | TT | GG |
| 189 | 38.6 | TT | TT | TT | AG | AG | AA | TT | GG | TT | GG |
| 190 | 38.7 | AT | AT | AT | AA | AA | AA | TT | GG | TT | GG |
| 191 | 38.7 | AT | AT | AT | AG | AG | AA | TT | GG | TT | GG |
| 198 | 39.0 | AA | AA | AA | AG | AG | AA | TT | GG | TT | GG |
| 209 | 39.3 | TT | TT | TT | AA | AA | AA | TT | GG | TT | GG |
| 214 | 39.5 | AA | AA | AA | AA | AA | AA | TT | GG | TT | GG |
| 218 | 39.6 | AT | AT | AT | AA | AA | AA | TT | GG | TT | GG |
| 222 | 39.7 | AT | TT | TT | GG | AG | AT | GT | GG | CT | AG |
| 246 | 40.3 | TT | AT | AT | AG | AG | AA | TT | GG | TT | GG |
| 252 | 40.6 | AT | AT | AT | AA | AA | AA | TT | GG | TT | GG |
| 255 | 40.8 | AT | AT | AT | AG | AG | AA | TT | GG | TT | GG |
| 262 | 41.0 | TT | TT | TT | AA | AA | AA | TT | GG | TT | GG |
| 278 | 41.4 | AT | TT | AT | GG | GG | AA | TT | GG | TT | GG |
| 283 | 41.5 | AA | AA | AA | GG | GG | AA | TT | GG | TT | GG |
| 284 | 41.5 | TT | TT | TT | GG | GG | AA | TT | GG | TT | GG |
| 287 | 41.5 | AT | AT | AT | AA | AA | AA | TT | GG | TT | GG |
| 288 | 41.5 | TT | TT | TT | AA | AA | AA | TT | GG | TT | GG |
| 289 | 41.5 | AA | AA | AA | AG | AG | AA | TT | GG | TT | GG |
| 293 | 41.6 | AT | TT | TT | GG | AG | AT | GT | GG | CT | AG |
| 299 | 41.9 | AA | AT | AA | AG | AG | AA | TT | GG | TT | GG |
| 304 | 42.0 | TT | TT | TT | AG | AG | AA | TT | GG | TT | GG |
| 305 | 42.0 | AT | AT | AT | AG | AG | AA | TT | GG | TT | GG |
| 320 | 42.5 | AA | AA | AA | GG | GG | AA | TT | GG | TT | GG |
| 321 | 42.5 | AA | AA | AA | AG | AG | AA | TT | GG | TT | GG |
| 322 | 42.6 | AT | AT | AT | AA | AA | AA | TT | GG | TT | GG |
| 323 | 42.6 | AT | AT | AT | AG | AG | AA | TT | GG | TT | GG |
| 324 | 42.6 | AA | AA | AA | AA | AA | AA | TT | GG | TT | GG |
| 326 | 42.6 | TT | TT | TT | AA | AA | AA | TT | GG | TT | GG |
| 331 | 42.9 | AT | TT | AT | AG | AG | AA | TT | GG | TT | GG |
| 335 | 43.0 | TT | TT | TT | AG | AG | AA | TT | GG | TT | GG |
| 343 | 43.3 | AA | AT | AA | AG | AA | AT | GT | GG | CT | AG |
| 350 | 43.6 | TT | TT | TT | AG | AG | AA | TT | GG | TT | GG |
| 353 | 43.6 | TT | TT | TT | AA | AA | AA | TT | GG | TT | GG |
| 356 | 43.7 | AT | AT | AT | AA | AA | AA | TT | GG | TT | GG |
| 360 | 43.8 | AA | AA | AA | AA | AA | AA | TT | GG | TT | GG |
| 363 | 43.8 | AT | TT | AT | AG | AG | AA | TT | GG | TT | GG |
| 378 | 44.3 | TT | AA | AA | GG | GG | AA | TT | GG | TT | GG |
| 386 | 44.5 | TT | TT | TT | AA | AA | AA | TT | GG | TT | GG |
| 388 | 44.5 | AT | AT | AT | AG | AG | AA | TT | GG | TT | GG |
| 396 | 44.7 | AT | AT | AT | AA | AA | AA | TT | GG | TT | GG |
| 428 | 45.3 | AT | TT | AT | AG | AG | AA | TT | GG | TT | GG |
| 430 | 45.4 | TT | TT | TT | GG | GG | AA | TT | GG | TT | GG |
| 432 | 45.5 | TT | TT | TT | AG | AG | AA | TT | GG | TT | GG |
| 433 | 45.5 | AT | AT | AT | AG | AG | AA | TT | GG | TT | GG |
| 436 | 45.5 | AT | AT | AT | AA | AA | AA | TT | GG | TT | GG |
| 442 | 45.6 | TT | TT | TT | GG | GG | AA | TT | GG | TT | GG |
| 445 | 45.7 | AA | AA | AA | AG | AG | AA | TT | GG | TT | GG |
| 459 | 46.0 | AA | AT | AA | AG | AG | AA | TT | GG | TT | GG |
| 466 | 46.2 | AA | AA | AA | GG | GG | AA | TT | GG | TT | GG |
| 469 | 46.2 | TT | TT | TT | AA | AA | AA | TT | GG | TT | GG |
| 471 | 46.3 | AT | AT | AT | GG | GG | AA | TT | GG | TT | GG |
| 476 | 46.5 | AA | AA | AA | AA | AA | AA | TT | GG | TT | GG |
| 477 | 46.5 | NN | TT | AT | AA | AA | AA | TT | NN | TT | GG |
| 479 | 46.6 | AT | AT | AT | AA | AA | AA | TT | GG | TT | GG |
| 481 | 46.6 | AT | AT | AT | AG | AG | AA | TT | GG | TT | GG |
| 483 | 46.6 | AT | TT | AT | AG | AG | AA | TT | GG | TT | GG |
| 484 | 46.6 | AA | AA | AA | AG | AG | AA | TT | GG | TT | GG |
| 489 | 46.7 | TT | TT | TT | AA | AA | AA | TT | GG | TT | GG |
| 490 | 46.7 | AT | TT | AT | GG | GG | AA | TT | GG | TT | GG |
| 523 | 47.5 | AT | AT | AT | AA | AA | AA | TT | GG | TT | GG |
| 530 | 47.8 | AT | TT | AT | AG | AG | AA | TT | GG | TT | GG |
| 532 | 47.8 | AT | AT | AT | AG | AG | AA | TT | GG | TT | GG |
| 534 | 47.9 | AA | AA | AA | AG | AG | AA | TT | GG | TT | GG |
| 546 | 48.4 | TT | TT | TT | AA | AA | AA | TT | GG | TT | GG |
| 553 | 48.6 | AT | AT | AT | AA | AA | AA | TT | GG | TT | GG |
| 554 | 48.6 | AA | AA | AA | AA | AA | AA | TT | GG | TT | GG |
| 557 | 48.7 | TT | TT | TT | AA | AA | AA | TT | GG | TT | GG |
| 558 | 48.7 | AT | AT | AT | AG | AG | AA | TT | GG | TT | GG |
| 560 | 48.7 | AT | TT | AT | AG | AG | AA | TT | GG | TT | GG |
| 562 | 48.8 | TT | TT | TT | AG | AG | AA | TT | GG | TT | GG |
| 589 | 49.3 | TT | TT | TT | GG | GG | AA | TT | GG | TT | GG |
| 592 | 49.5 | AT | AT | AT | AA | AA | AA | TT | GG | TT | GG |
| 593 | 49.5 | AT | TT | TT | AG | AA | AT | GT | GG | CT | AG |
| 594 | 49.6 | AT | AT | AT | AG | AG | AA | TT | GG | TT | GG |
| 595 | 49.6 | AT | TT | AT | AG | AG | AA | TT | GG | TT | GG |
| 596 | 49.6 | AA | AA | AA | AG | AG | AA | TT | GG | TT | GG |
| 601 | 49.7 | TT | TT | TT | AG | AG | AA | TT | GG | TT | GG |
| 604 | 49.8 | TT | TT | AT | AA | AA | NN | TT | NN | CT | GG |
| 616 | 50.2 | NN | TT | TT | AG | GG | NN | TT | NN | TT | GG |
| 630 | 50.6 | TT | TT | TT | AA | AA | AA | TT | GG | TT | GG |
| 631 | 50.6 | TT | TT | TT | AG | AG | AA | TT | GG | TT | GG |
| 633 | 50.6 | AA | AA | AA | AA | AA | AA | TT | GG | TT | GG |
| 651 | 50.9 | AT | AT | AT | AA | AA | AA | TT | GG | TT | GG |
| 658 | 51.2 | AA | AA | AA | AG | NN | AT | TT | GG | TT | GG |
| 669 | 51.6 | TT | TT | TT | AA | AA | AA | TT | GG | TT | GG |
| 670 | 51.6 | AA | AA | AA | AG | AG | AA | TT | GG | TT | GG |
| 672 | 51.6 | AT | AT | AT | AG | AG | AA | TT | GG | TT | GG |
| 676 | 51.8 | AA | AA | AA | AA | AA | AA | TT | GG | TT | GG |
| 678 | 51.9 | AT | AT | AT | GG | GG | AA | TT | GG | TT | GG |
| 699 | 52.5 | AT | AT | AT | AA | AA | AA | TT | GG | TT | GG |
| 700 | 52.5 | AT | AT | AT | AG | AG | AA | TT | GG | TT | GG |
| 701 | 52.6 | AA | AA | AA | AA | AA | AA | TT | GG | TT | GG |
| 702 | 52.6 | TT | TT | TT | AG | AG | AA | TT | GG | TT | GG |
| 703 | 52.7 | TT | TT | TT | AA | AA | AA | TT | GG | TT | GG |
| 707 | 52.9 | AA | AA | AA | AG | AG | AA | TT | GG | TT | GG |
| 718 | 53.2 | AA | AA | AA | GG | GG | AA | TT | GG | TT | GG |
| 726 | 53.5 | AT | AT | AT | AG | AG | AA | TT | GG | TT | GG |
| 729 | 53.6 | AT | AT | AT | AA | AA | AA | TT | GG | TT | GG |
| 746 | 54.0 | TT | TT | TT | AG | AG | AA | TT | GG | TT | GG |
| 749 | 54.1 | AA | AA | AA | AA | AA | AA | TT | GG | TT | GG |
| 754 | 54.5 | AT | TT | AT | AG | AG | AA | TT | GG | TT | GG |
| 759 | 54.5 | AT | AT | AT | GG | GG | AA | TT | GG | TT | GG |
| 764 | 54.7 | AA | AA | AA | AG | AG | AA | TT | GG | TT | GG |
| 765 | 54.8 | TT | TT | TT | AA | AA | AA | TT | GG | TT | GG |
| 767 | 55.0 | AT | AT | AT | AA | AA | AA | TT | GG | TT | GG |
| 779 | 55.6 | AA | AA | AA | AG | AG | AA | TT | GG | TT | GG |
| 780 | 55.6 | AT | AT | AT | AG | AG | AA | TT | GG | TT | GG |
| 782 | 55.8 | TT | TT | TT | AA | AA | AA | TT | GG | TT | GG |
| 789 | 56.0 | AT | TT | AT | GG | GG | AA | TT | GG | TT | GG |
| 796 | 56.3 | AT | AT | AT | GG | GG | AA | TT | GG | TT | GG |
| 798 | 56.5 | AT | AT | AT | AA | AA | AA | TT | GG | TT | GG |
| 804 | 56.8 | TT | TT | TT | AA | AA | AA | TT | GG | TT | GG |
| 807 | 57.1 | AT | AT | AT | AG | AG | AA | TT | GG | TT | GG |
| 829 | 58.7 | AA | AA | AA | AA | AA | AA | TT | GG | TT | GG |
| 830 | 58.7 | AT | AT | AT | AA | AA | AA | TT | GG | TT | GG |
| 841 | 59.3 | AT | AT | AT | GG | GG | AA | TT | GG | TT | GG |
| 846 | 59.7 | AA | AA | AA | AG | AG | AA | TT | GG | TT | GG |
| 849 | 59.8 | AA | AT | AA | AG | AG | AA | TT | GG | TT | GG |
| 850 | 59.9 | AT | TT | AT | AG | AG | AA | TT | GG | TT | GG |
| 851 | 59.9 | AA | AA | AA | AA | AA | AA | TT | GG | TT | GG |
| 852 | 59.9 | AT | AT | AT | AG | AG | AA | TT | GG | TT | GG |
| 855 | 60.3 | TT | TT | TT | AA | AA | AA | TT | GG | TT | GG |
| 863 | 61.6 | AT | AT | AT | AA | AA | AA | TT | GG | TT | GG |
| 868 | 62.5 | AA | AT | AA | AG | AG | AA | TT | GG | TT | GG |
| 872 | 62.9 | AT | AT | AT | AA | AA | AA | TT | GG | TT | GG |
| 877 | 63.2 | AA | AA | AA | AG | AG | AA | TT | GG | TT | GG |
| 893 | 64.6 | TT | TT | TT | AG | AG | AA | TT | GG | TT | GG |
| 898 | 65.6 | TT | TT | TT | AA | AA | AA | TT | GG | TT | GG |
| 903 | 66.4 | AT | AT | AT | AA | AA | AA | TT | GG | TT | GG |
| 904 | 66.5 | AT | AT | AT | AG | AG | AA | TT | GG | TT | GG |
| 909 | 66.9 | AA | AA | AA | AA | AA | AA | TT | GG | TT | GG |
| 910 | 67.4 | AT | AT | AT | AA | AA | NN | GT | GG | CT | NN |
| 913 | 67.5 | AT | AT | AT | AA | AA | AA | TT | GG | TT | GG |
| 914 | 67.7 | TT | TT | TT | AA | AA | AA | TT | GG | TT | GG |
| 918 | 68.8 | AT | AT | AT | AA | AA | AA | TT | GG | TT | GG |
| 920 | 69.5 | AT | AT | AT | AG | AG | AA | TT | GG | TT | GG |
| 921 | 69.6 | AA | AA | AA | GG | GG | AA | TT | GG | TT | GG |
| 923 | 69.8 | AA | AA | AA | AG | AG | AA | TT | GG | TT | GG |
| 924 | 70.3 | AT | AT | AT | AA | AA | AA | TT | GG | TT | GG |
| 925 | 70.6 | TT | TT | TT | AA | AA | AA | TT | GG | TT | GG |
| 928 | 71.7 | AA | AA | AA | AG | AG | AA | TT | GG | TT | GG |
| 931 | 72.6 | AT | AT | AT | AA | AA | AA | TT | GG | TT | GG |
| 932 | 73.1 | TT | TT | TT | AG | AG | AA | TT | GG | TT | GG |
| 934 | 73.6 | AA | AA | AA | AA | AA | AA | TT | GG | TT | GG |
| 939 | 74.8 | AT | AT | AT | AG | AG | AA | TT | GG | TT | GG |
| 944 | 76.1 | AA | AT | AA | AG | AG | AA | TT | GG | TT | GG |
| 947 | 76.6 | TT | TT | TT | AA | AA | AA | TT | GG | TT | GG |
| 949 | 78.1 | AA | AA | AA | GG | GG | AA | TT | GG | TT | GG |
| 952 | 79.9 | AT | AT | AT | AA | AA | AA | TT | GG | TT | GG |
| 954 | 81.7 | AA | AA | AA | AA | AA | AA | TT | GG | TT | GG |
| 957 | 84.1 | AT | AT | AT | AG | AG | AA | TT | GG | TT | GG |
| 960 | 86.3 | AT | AT | AT | AA | AA | AA | TT | GG | TT | GG |
| 963 | 89.4 | TT | TT | TT | AA | AA | AA | TT | GG | TT | GG |
| 980 | 93.0 | AT | AT | AT | AG | AG | AA | TT | GG | TT | GG |
| 969 | 93.3 | AT | TT | AT | AG | AG | AA | TT | GG | TT | GG |
| 970 | 94.0 | AT | AT | AT | AA | AA | AA | TT | GG | TT | GG |
| | WT | TT | TT | TT | AA | AA | AA | TT | GG | TT | GG |
| | HT | AT | AT | AT | AG | AG | AT | GT | CG | CT | AG |
| | MT | AA | AA | AA | GG | GG | TT | GG | CC | CC | AA |

Table 8 gives a detailed summary of haplotypes that are correlated to TPMT enzyme activity. Of special interests are haplotypes that identify absent, low or medium TPMT enzyme activity.

TABLE 8
Detailed Summary of Haplotypes correlated to TPMT Enzyme Activity

| Hap 1 | SNP26 | | SNP29 | Activity |
|---|---|---|---|---|
| | MT | and | WT | 0 |
| | HT | and | WT | 1 |
| | MT | and | HT | 1 |
| | MT | and | MT | 2 |
| | HT | and | HT | 2 |
| | HT | and | MT | 2 |
| | WT | and | WT | 2 |

| Hap 2 | SNP20 | | SNP7 | | SNP8 | SNP26 | SNP29 | Activity |
|---|---|---|---|---|---|---|---|---|
| | MT | and | MT | and | WT and | WT and | WT | 0 |
| | HT | and | MT | and | WT | | | 1 |
| | WT | and | WT | and | HT or MT | | | 1 |
| | MT | and | MT | and | MT | | | 2 |
| | HT | and | HT | and | HT | | | 2 |
| | WT | and | HT | and | HT | | | 2 |
| | WT | and | MT | and | MT | | | 2 |
| | WT | and | WT | and | WT | | | 2 |

| Hap 3 | SNP10 | | SNP17 | Activity |
|---|---|---|---|---|
| | MT | and | MT | 0 |
| | HT or MT | and | WT | 1 |
| | MT | and | HT | 1 |
| | HT or WT | and | HT | 1 |
| | WT | and | MT | 1 |
| | WT | and | WT | 2 |

| Reference SNPs | SNP44 | | SNP47 | | SNP50 | Activity |
|---|---|---|---|---|---|---|
| | HT | or | MT | and | MT | 0 |
| | | | HT | and/or | HT | 1 |
| | | | HT | and | MT | 1 |
| | | | MT | or | MT | 1 |
| | | | WT | and | WT | 2 |

The corresponding genotype of each SNP being WT,
HT or MT can be read from the following table:

| | SNP20 | SNP8 | SNP7 | SNP26 | SNP29 | SNP10 | SNP17 | SNP44 | SNP47 | SNP50 |
|---|---|---|---|---|---|---|---|---|---|---|
| WT | TT | TT | TT | AA | AA | AA | TT | GG | TT | GG |
| HT | AT | AT | AT | AG | AG | AT | GT | CG | CT | AG |
| MT | AA | AA | AA | GG | GG | TT | GG | CC | CC | AA |

Legend of Table 8: SNP genotypes: WT = wildtype; HT = heterozygote; MT = mutant TPMT enzyme activity: 0 = absent or low, 1 = medium; 2 = normal or high

A descriptive summary of the best haplotypes that are correlated to absent, low or medium TPMT activity are given below:

### Haplotype Group 1:

When SNP 26 being MUTANT and SNP 29 being WILDTYPE the TPMT enzyme activity is absent or low.

When SNP 26 being HETEROZYGOTE and SNP 29 being WILDTYPE the TPMT enzyme activity is medium.

When SNP 26 being MUTANT and SNP 29 being HETEROZYGOTE the TPMT enzyme activity is medium.

### Haplotype Group 2:

When SNP 20 being MUTANT and SNP 7 being MUTANT and SNP 8 being WILDTYPE and SNP 26 being WILDTYPE and SNP 29 being WILDTYPE the TPMT enzyme activity is absent or low.

When SNP 20 being HETEROZYGOTE and SNP 7 being MUTANT and SNP 8 being WILDTYPE the TPMT enzyme activity is medium.

When SNP 20 being WILDTYPE and SNP 7 being WILDTYPE and SNP 8 being HETEROZYGOTE or MUTANT the TPMT enzyme activity is medium.

### Haplotype Group 3:

When SNP 10 being MUTANT and SNP 17 being MUTANT the TPMT enzyme activity is absent or low.

When SNP 10 being HETEROZYGOTE or MUTANT and SNP 17 being WILDTYPE the TPMT enzyme activity is medium.

When SNP 10 being MUTANT and SNP 17 being HETEROZYGOTE the TPMT enzyme activity is medium.

When SNP 10 being HETEROZYGOTE or WILDTYPE and SNP 17 being HETEROZYGOTE the TPMT enzyme activity is medium.

When SNP 10 being WILDTYPE and SNP 17 being MUTANT the TPMT enzyme activity is medium.

As a further embodiment of this invention one can combine the predictive power of the here described genotype and haplotype correlations to the TPMT expression with the number of VNTRs in the respective patients. Whereas a higher number of repeats responds inversely to the TPMT activity.

1. An isolated polynucleotide molecule comprising a mutant allele of thiopurine S-methyltransferase (TPMT) gene or fragments thereof containing single nucleotide polymorphisms (SNPs 1-41) as shown in Table 1.

2. An isolated polynucleotide molecule comprising a mutant allele of thiopurine S-methyltransferase (TPMT) gene or a fragment thereof containing at least two or more of single nucleotide polymorphisms (SNPs 1-41) as shown in Table 1.

3. An isolated polynucleotide molecule comprising a mutant allele of thiopurine S-methyltransferase (TPMT) gene or fragments thereof containing single nucleotide polymorphisms, SNPs 10 and/or 17, and/or 26 and 29 in the following haplotypes (combinations):
a) SNP 26 being MT (GG) and SNP 29 being WT (GG)
b) SNP 26 being HT (AG) and SNP 29 being WT (GG)
c) SNP 26 being MT (GG) and SNP 29 being HT (AG)
d) SNP 10 being MT (TT) and SNP 17 being MT (GG)
e) SNP 10 being HT (AT) or MT (TI) and SNP 17 being WT (TIT)
f) SNP 10 being MT (TT) and SNP 17 being HT (GT)
g) SNP 10 being HT (AT) or WT (AA) and SNP 17 being HT (GT)
h) SNP 10 being WT (AA) and SNP 17 being MT (GG).

4. An isolated polynucleotide molecule comprising a mutant allele of thiopurine S-methyltransferase (TPMT) gene or fragments thereof containing single nucleotide polymerphisms, SNPs 7, 8, 20 and/or 26 and 27 in the following haplotypes (combinations):
a) SNP 7 being MT (AA) and SNP 8 being WT (TT) and SNP 20 being MT (AA) and SNP 26 being WT (AA) and SNP 29 being WT (AA)
b) SNP 7 being MT (AA) and SNP 8 being WT (TT) and SNP 20 being HT (AT)
c) SNP 7 being WT (TT) and SNP 8-being HT (AT) or MT (AA) and SNP 20 being WT (TT).

5. An isolated polynucleotide molecule fully complementary to any one of the polynucleotide molecules of claims 1-4.

6. A diagnostic assay or kit for determining thiopurine S-methyl-trasferase (TPMT) genotype of a subject which comprises
a) isolating nucleic acid from said subject;
b) amplifying specifically a thiopurine S-methyltransferase (TPMT) PCR fragment with primers of Table 2 from said nucleic acid, which includes at least one of SNPs of claims 1-4 thereby obtaining an amplified fragment; and
c) genotyping the amplified fragment obtained in step b), thereby determining the thiopurine S-methyltransferase (TPMT) genotype or haplotype of said subject,
d) the kit comprising sequence determination primers and sequence determination reagents, wherein said primers are selected from the group comprising primers that hybridize to polymorphic positions in the human TPMT genes according to claims 1-4; and primers that hybridize immediately adjacent to polymorphic positions in the human TPMT gene according to claims 1-4.

7. A kit as defined in claim 6 detecting a combination of two or more, up to all, polymorphic sites selected from the groups of sequences as defined in claim 1-4.

8. A method for determining a patient's individual response to thiopurine therapy, including drug efficacy and adverse drug reactions, comprising determining the identity of nucleotide variations according to claims 1-4.
